(19) United States
(12) Patent Application Publication
(10) Pub. No.: US 2009/0136547 A1
(43) Pub. Date: May 28, 2009

(54) ZWITTERIONIZATION OF CAPSULAR SACCHARIDES

(75) Inventors: John Telford, Siena (IT); Francesco Berti, Siena (IT); Andreas Wack, Siena (IT)

Correspondence Address: NOVARTIS VACCINES AND DIAGNOSTICS INC., INTELLECTUAL PROPERTY R338, P.O. BOX 8097, Emeryville, CA, 94662-8097, US

(73) Assignee: NOVARTIS VACCINES AND DIAGNOSTICS SRL, Siena (IT)

(21) Appl. No.: 12/064,663

(22) PCT Filed: Aug. 24, 2006
(86) PCT No.: PCT/IB2006/002833

§ 371 Date: Aug. 25, 2008

(30) Foreign Application Priority Data
Aug. 24, 2005 (GB) 0517353.9
Apr. 19, 2006 (GB) 0607738.2

(51) Int. Cl. A61K 39/09; C08B 37/00; A61K 39/095; A61K 39/02; A61P 31/04

(52) U.S. Cl. 424/244.1; 536/123.1; 424/250.1; 424/234.1

(57) ABSTRACT

Capsular saccharides are typically anionic. In the invention, however, cationic groups are introduced, such that the modified saccharide has a repeating unit which includes both cationic and anionic groups. These cationic and anionic groups can be balanced to give a zwitterionic repeating unit. These modifications can convert a saccharide that is normally a T-independent antigen into one that can activate T cells without requiring conjugation to a carrier. Typically, the invention modifies an anionic bacterial capsular saccharide antigen by converting a neutral group in the saccharide into a cationic group e.g. to change —NHAc to —NH₃⁺.

All documents cited herein are incorporated by reference in their entirety.

### TECHNICAL FIELD

This invention is in the field of vaccines based on saccharide antigens.

### BACKGROUND ART

Bacterial capsular saccharides are generally T-independent antigens, but they can be converted to T-dependent antigens by conjugating them to carrier proteins. It is an object of the invention to provide alternatives to conjugation for making saccharides into T-dependent antigens, and more generally to provide ways of modifying saccharides so that they can activate T cells.

### DISCLOSURE OF THE INVENTION

Capsular saccharides are typically anionic, as a result of an otherwise neutral sugar backbone carrying anionic groups such as carboxyls (which are present as carboxylate ions at physiological pH). In the invention, however, a T-independent bacterial capsular saccharide antigen is converted into a saccharide that can activate T cells (e.g. a T-dependent saccharide antigen) by modifying it to include both anionic and cationic groups. In preferred embodiments, the anionic and cationic groups are balanced with the saccharide's repeating unit, to give a zwitterion. Zwitterionic capsular saccharide antigens from Bacteroides fragilis have been found to stimulate T cells by MHC class II-dependent interactions [1], and similar stimulation has been seen for Staphylococcus aureus and type 1 Streptococcus pneumoniae. Without wishing to be bound by theory, the invention aims to mimic this MHC-II stimulatory effect for saccharides that are naturally T-independent, thereby rendering them immunogenic without requiring conjugation to a carrier.

Thus the invention provides a method for modifying an anionic bacterial capsular saccharide antigen, comprising a step of converting a neutral group in the saccharide into a cationic group.

The invention also provides a method for modifying a cationic bacterial capsular saccharide antigen, comprising a step of converting a neutral group in the saccharide into an anionic group.

The invention also provides a method for modifying a neutral bacterial capsular saccharide antigen, comprising steps of (i) converting a first neutral group in the saccharide into an anionic group and (ii) converting a second neutral group in the saccharide into a cationic group.

For convenience, these various methods are referred to as ‘zwitterionization’, even though the final modified repeating unit may not formally be a zwitterion. For example, the type 1 S. pneumoniae saccharide (FIG. 12) is generally referred to as being a zwitterion even though it has an overall negative charge. The key point is that the modified saccharide has a repeating unit which includes both cationic and anionic groups, which may or may not be balanced to give a zwitterion. Preferably, however, the charges are balanced such that the repeating unit is a zwitterion.

The invention also provides a modified bacterial capsular saccharide, wherein the saccharide in its natural form includes repeating units that are cationic, but the saccharide in its modified form includes repeating units that are zwitterionic or anionic. The repeating units in the modified saccharide are preferably zwitterionic.

The invention also provides a modified bacterial capsular saccharide, wherein the saccharide in its natural form includes repeating units that are anionic, but the saccharide in its modified form includes repeating units that are zwitterionic or cationic. The repeating units in the modified saccharide are preferably zwitterionic.

The invention also provides a modified bacterial capsular saccharide, wherein the saccharide in its natural form includes repeating units that include either cationic or anionic groups (but not both), but the saccharide in its modified form includes repeating units that include both cationic and anionic groups. The repeating units in the modified saccharide are preferably zwitterionic.

The invention also provides a modified bacterial capsular saccharide, wherein the saccharide includes a repeating unit that (i) includes both positively-charged and negatively-charged groups but (ii) has no overall charge.

Preferred repeating units have both free carboxyl groups and free amino groups, thereby providing both positively-charged and negatively-charged groups, preferably in an overall neutral zwitterionic repeating unit.

The invention is particularly suitable for use with capsular saccharides from (a) group B streptococcus (‘GBS’, or Streptococcus agalactiae), including serotypes Ia, Ib, II, III and V, and (b) meningococcus (Neisseria meningitidis), including each of meningococcal serogroups A, B, C, W135 and Y. These saccharides are naturally polyanionic (i.e. they have multiple anionic repeating units, typically carboxyls) but can be chemically modified by introducing positively-charged groups, preferably into a zwitterionic form. For instance, neutral N-acetyl groups can be converted to amino groups able to carry positive charge as —NH₃⁺.

### Zwitterionization

A zwitterion is a molecule that has both positively-charged and negatively-charged groups but has no overall charge i.e. the + and − charges are balanced within the molecule. To convert an anionic molecule into a neutral molecule then anions would typically be replaced by neutral groups, but to convert an anionic molecule into a zwitterion then a neutral group is replaced by a cationic group. According to the invention, the term ‘zwitterionization’ includes situations where a saccharide with a repeating unit that includes only anionic or cationic groups (but not both), or that includes no charged groups, is modified to have a repeating unit that includes both cationic and anionic groups. The modified repeating unit may have neutral overall charge (i.e. may be a zwitterion), but the invention also extends to situations where the charges are present but are not balanced. The modified repeating unit may be amphoteric (i.e. can react with an acid or a base).

Once a naturally-charged capsular saccharide has been selected for zwitterionization, an initial step in the process involves the identification of a charged repeating unit. The charge in an anionic repeating unit will typically result from the presence of free carboxyl groups [—COO⁻]. A neutral group in the repeating unit is identified, and in particular a neutral group that can be converted to a group having the opposite charge to the unmodified repeating unit i.e. for an anionic repeating unit, a neutral group that can be converted to a cation is identified. The positive charge in the modified repeating unit will typically result from the presence of protonated free amino groups [—NH₃⁺], which can conveniently be prepared from free N-acetyl groups [—NH—(C═O)—CH₃]. To introduce an anionic group, a carboxyl can be introduced, for instance by conversion of —CH₂OH to —COO⁻ (optionally via an aldehyde group).

The ability to convert a neutral group into an ionic group is thus a key aspect of zwitterionization. For anionic saccharides, which is the most common type of bacterial capsular saccharide, the ability to change a neutral group to a cationic group is important. Typical cations for use with the invention are protonated forms of primary amines (amino groups —NH₂, which can be protonated to give cationic —NH₃⁺), secondary amines (—NRH→—NRH₂⁺) and tertiary amines (—NR¹R²→—NR¹R²H⁺). Cationic quaternary amines (—NR¹R²R³⁺) can also be used, such as —N(CH₃)₃⁺. These groups can conveniently be prepared from free N-acetyl groups, which are frequently found in capsular saccharides as part of sialic acid (N-acetylneuraminic acids, ‘NeuNAc’; FIG. 4) and N-acetylglucosamine (‘GlcNAc’) residues. The nitrogen atom in a N-acetyl group does not readily accept a proton at physiological pH and so, for zwitterionization, N-acetyl groups can be replaced with N-linked groups with a lower pK_{b} value, such as the amino and amine groups discussed above. Amino groups can also be introduced in place of existing uncharged hydroxyl groups (e.g. to make an amino sugar), which can be used instead of de-N-acetylation if the N-acetyl group is important for immunoreactivity.

For introduction of a cationic group, therefore, a neutral group should be converted to a group with a lower pK_{b} value (i.e. to a stronger base). Conversely, where an anionic group is to be introduced then a neutral group should be converted to a group with a lower pKₐ (i.e. higher pK_{b}) value.

As mentioned above, cationic groups that can easily be generated from existing neutral groups include primary amine, secondary amine, tertiary amine and amino groups.

Anionic groups that can easily be introduced during zwitterionization include phosphates, phosphonates, sulfates, nitrates and carboxyls.

The most preferred cationic group to introduce during zwitterionization is a free (i.e. unsubstituted) amino group. The free amino group may then be substituted as discussed above. The most preferred anionic group to introduce during zwitterionization is a free carboxyl group.

Examples of chemical treatments to remove or introduce charged groups in bacterial capsular saccharides are outlined in FIG. 11. These schemes are shown for GBS-III, but are equally applicable to other GBS serotypes, including Ia and Ib.

In general, de-N-acetylation can be achieved by basic hydrolysis to reveal free amino groups. As shown in scheme I in FIG. 11, a N-acetyl group (either in an internal residue or a terminal residue) can be converted first (step Ia) to an amine by de-N-acetylation (e.g. via NaOH treatment) and the amine can then be converted to a cationic —NH₃^{+ group. If desired, scheme II can then be used to convert the amino group to a tertiary amine (for example, to tertiary dimethylamine —NH(CH}₃)₂⁺) e.g. by formaldehyde treatment under suitable conditions (such as 37% formaldehyde) in step Ha. These higher amines are more stable e.g. resistant to the effects of pH changes.

Scheme III in FIG. 11 illustrates removal of an anionic carboxyl. A carboxyl group can be associated with pyruvate (—COCOOCH₃) and reduced by carbodiimide-mediated reduction. The pyruvate ring can also be removed by treatment with acetic acid (e.g. 5%). Further modifications of an introduced pyruvate residue are possible. For example, step IIIb reduces the carboxyl to an aldehyde e.g. using NaBH₄. The carboxyl can also be reduced to an aldehyde by using SOCl₂ or a phosphorus chloride reagent such as phosphorus trichloride or phosphorus pentachloride to form an acid chloride. The acid chloride can then be reduced to an aldehyde by using a suitable reducing agent, which is preferably a hydride donor, e.g. LiAlH₄ or sodium borohydride. The aldehyde can then be reduced to an alcohol in step IIIc e.g. using sodium borohydride. The removal of an anionic carboxyl can also be achieved by treatment of the anionic carboxyl group with a reagent such as EDC (1-ethyl-3-(3-dimethylaminopropyl)carbodiimide) and sodium borohydride. Alternatively, the anionic carboxyl can be reduced to an alcohol in a single step by using LiAlH₄ or BH₃ for example. Scheme III can be used after basic de-N-acetylation. As the effect of modification III is to remove an anionic group, this procedure will typically be used in conjunction with other modifications. The modification can be used in a procedure to move a negative charge to another site in a repeating unit e.g. to give a desired spacing of negative and positive charges.

As shown in scheme IV in FIG. 11, glycosidic linkages in saccharides can be cleaved by various treatments, including enzymatic (e.g. with β-endogalactosidase) or chemical (e.g. ozonolysis, nitric oxide treatment, acid hydrolysis, base hydrolysis, etc.) treatments. These depolymerization treatments can leave terminal groups that can be used during zwitterionization. For example, FIG. 11 shows an initial fragmentation in step IVa using ozonolysis, nitric oxide or β-gal treatment. Step IVb oxidises the end of the fragmented saccharide to generate a terminal aldehyde e.g. using galactose oxidase. Step IVc then converts the aldehyde to a cationic —NH₃⁺ group by reductive amination e.g. using ammonium and sodium cyanoborohydride. Reductive amination may involve either ammonia or a primary amine (NHR). This can conveniently be achieved by using an ammonium salt (e.g. ammonium chloride) in combination with an appropriate reducing agent (e.g. cyanoborohydrides, such as sodium cyanoborohydride NaBH₃CN; borane-pyridine; sodium triacetoxyborohydride; borohydride exchange resin; etc.). After its introduction, an amino can be further converted to a secondary or tertiary amine as described above for schemes I & II (e.g. to tertiary dimethylamine —NH(CH₃)₂⁺). Scheme IV may be used particularly after a saccharide has been subjected to total (re-)N-acetylation of its residues.

Scheme V in FIG. 11 begins by removing the aliphatic chain (—CHOH—CH₂OH) from a terminal NeuNAc residue (e.g. by mild oxidation, as achieved using sodium periodate at low temperature) to leave —CHO (i.e. the C9 carbon is lost). This aldehyde group can then be converted to a cationic group (e.g. —NH₃⁺, or higher amine) as described for scheme IV. Scheme V may be used particularly in situations where it is desired to maintain N-acetyl groups e.g. after a saccharide has been subjected to total (re-)N-acetylation of its residues.

Scheme VI in FIG. 11 converts a hydroxyl (—CH₂OH) to a carboxyl (—COO⁻), thereby replacing a neutral group with an anionic group. The conversion can be achieved using a strong oxidising agent, such as H₂O₂, KMnO₄ or H₂CrO₄, in step Via. Conversion from hydroxyl to carboxyl can also be achieved (scheme VII) via an aldehyde intermediate e.g. an initial Va with further oxidation in step Vila. Selective conversion of a primary hydroxyl (—CH₂OH) to a carboxyl (—COO⁻) can be achieved by use of TEMPO (2,2,6,6-tetramethyl-1-piperidine oxoammonium ion) in the presence of hypochlorite and bromide.

In general, aldehydes can readily be prepared by oxidizing vicinal hydroxyls groups (diols). The aldehyde can then be converted to carboxyl by further oxidation step e.g. with H₂O₂. With reference to FIG. 11, the susceptible diols are, in order of reactivity (see also FIG. 34):
1) C8-C9 of the NeuAc residue
2) C7-C8 of the NeuAc residue;
3) C2-C3 of the Glucose residue.

These diols can be converted into aldehydes using reagents such as TEMPO and NaIO₄. The non-diol hydroxyls (e.g. the hydroxyl groups on C-6 of glucose moieties and on C-6 of galactose moieties) are typically not reactive with TEMPO or NaIO₄ but can be oxidized instead with, for example, KMnO₄, H₂O₂ or H₂CrO₄. Selective reaction of diolic hydroxyls and non-diolic hydroxyls can be achieved based on stoichiometry and/or the use of protection and de-protection of primary and secondary hydroxyl groups as necessary.

Further suitable chemical treatments are also disclosed in reference 2, including (a) modification of —NH₃⁺ groups to —NHAc or to —NH(CH₃)₂⁺, and (b) reduction of anionic —COO⁻ to —CH₂OH.

The balanced negative and positive charges in a zwitterionic repeating unit of the invention preferably arise from carboxyl and amino groups.

Neutral groups selected for modification during zwitterionisation are preferably not critical to the antigenic activity of the native saccharide. Thus, for instance, the modification should not remove the ability of a saccharide to induce a neutralizing antibody.

If the spacing of positive and negative groups in a zwitterionised repeating unit is important for a molecule's ability to act as a T-dependent antigen, it may be preferred that positive and negative charges should be on different monosaccharides within a repeating unit. However, it is also possible to have the charges on the same monosaccharide (e.g. the use of scheme V, shown in FIG. 11, gives a modified NeuNAc residue with a cationic amine and an anionic carboxyl). The repeating unit of the zwitterionized repeating unit may be different from the repeating unit of the natural saccharide. For example, if a saccharide is naturally a homopolymer (e.g. a polymer of anionic monosaccharides, . . . -N-N-N-N-N-N-N-N- , , , ) then it is possible to introduce alternating positive and negative charges along the chain (e.g. . . . -N-P-N-P-N-P-N-P- . . . ), such that the repeating unit becomes a disaccharide rather than a monosaccharide.

Positive and negative charges need not be on adjacent monosaccharides within a repeating unit. For instance, reference 3 teaches that, in a saccharide having a tetrasaccharide backbone -(M₁-M₂-M₃-M₄)-, M₁ should be anionic and M₄ should be cationic. According to reference 3, the cationic groups on M₄ units of adjacent tetrasaccharides are preferably less than 32 Å from each other, measured along the backbone. This distance will typically be between the nitrogens of free amino moieties.

Where a number of groups can be chosen for modification during zwitterionization then it is preferred to choose those which together result in suitable antigenic activity and T eel 1 activating activity.

Preferred zwitterionic repeating units can adopt a helical conformation, and in particular a right-handed helix. The helix may have about 10 monosaccharide units in the saccharide backbone for every turn of the helix [4].

Modified saccharides of the invention include at least one repeating unit that has been zwitterionised, whereas that repeating unit is cationic or anionic in the natural saccharide. The saccharide as a whole may be charged or may be neutral. An overall charge may result from the presence of non-zwitterionic repeating units in the saccharide as a whole. Preferably, however, at least 50% (e.g. ≧60%, ≧70%, ≧80%, ≧90%, ≧95%, ≧98%, or more) of the repeating units in a saccharide are zwitterionised. Overall neutrality may result from the presence of 100% zwitterionic repeating units, or from having, in addition to the zwitterionic repeating units, balanced non-zwitterionic repeating units (e.g. the 9-mers P-P-P-Z-Z-Z-N-N-N and P-Z-P-Z-N-P-N-Z-N where each P is a positive repeating unit, each Z is a zwitterionic repeating unit, and each N is a negative repeating unit). Incomplete zwitterionization of a long saccharide can occur, for instance, when only a portion of the repeating units are acetylated.

Zwitterions made using the invention should be neutral under physiological conditions. At lower or higher pH values then a zwitterion will acquire or lose protons, but it preferably has an isoelectric point (pI) between 6 and 8, more preferably about 7. For saccharides processed via intracellular endocytic compartments, a pI between 4 and 6 (e.g. about 5 or about 5.5) may be preferred.

The saccharide to be zwitterionized

Preferred saccharides for zwitterionization are those than contain sialic acid residues, optionally also including N-acetyl-containing residues other than sialic acid residues (e.g. GlcNAc residues). Thus GBS and meningococcal capsular saccharides are suitable for zwitterionization.

The capsular polysaccharides of different GBS serotypes are chemically related, but are antigenically very different. All GBS capsular polysaccharides share the following trisaccharide core:

β-D-GlcpNAc(1→3)β-D-Galp(1→4)β-D-Glcp

The various GBS serotypes differ by the way in which this core is modified. The difference between serotypes Ia and III, for instance, arises from the use of either the GlcNAc (Ia) or the Gal (III) in this core for linking consecutive trisaccharide cores (FIG. 2). Serotypes Ia and Ib both have a [α-D-NeupNAc(2→3)β-D-Galp-(1→] disaccharide linked to the GlcNAc in the core, but the linkage is either 1→4 (Ia) or 1→3 (Ib).

GBS-related disease arises primarily from serotypes Ia, Ib, II, III, IV, V, VI, VII, and VIII, with over 90% being caused by five serotypes: Ia, Ib, II, III & V. Zwitterionization is preferably used for one of these five serotypes, although can also be applied to the others. As shown in FIG. 1, the capsular saccharides of each of these five serotypes include: (a) a terminal N-acetyl-neuraminic acid (NeuNAc) residue (commonly referred to as sialic acid), which in all cases is linked 2→3 to a galactose residue; and (b) a N-acetyl-glucosamine residue (GlcNAc) within the trisaccharide core.

All five saccharides include galactose residues within the trisaccharide core, but serotypes Ia, Ib, II & III also contain additional galactose residues in each repeating unit, with the serotype II saccharide containing three galactose residues per repeating unit.

For each of these five GBS serotypes, there is a terminal NeuNAc residue and a non-terminal GlcNAc residue. The terminal NeuNAc provides a native anionic —COO⁻ group. For zwitterionization an available N-acetyl group can conveniently be cleaved to give an amino group. The N-acetyl on the terminal NeuNAc can be used, but to increase spacing between the zwitterion's positive and negative groups then it is preferred to use the N-acetyl group on the GlcNAc residue. This modification is illustrated in FIG. 3 for a repeating unit of the GBS-III saccharide.

The capsular saccharide of serogroup A meningococcus is shown in FIG. 5. It is (α1→6)-linked N-acetyl-D-mannosamine-1-phosphate homopolymer, with partial N- and O-acetylation. The main glycosidic bond is a 1,6-phosphodiester bond involving the hemiacetal group of C-1 and the alcohol group of C-6 of the D-mannosamine. Each repeating unit includes an anionic phosphate, so zwitterionization will generally involve the conversion of a non-phosphate group into a cation, such as conversion of the N-acetyl group on C-2 into an amino or amine group.

The capsular saccharide of serogroup B meningococcus is a homopolymer of sialic acid, linked α-2,8 with partial N-acetylation but no O-acetylation: poly(α2→8)NeuNAc, as shown in FIG. 6. Each repeating unit in the native saccharide includes an anionic carboxyl, so zwitterionization will generally involve the conversion of a non-carboxyl group into a cation, such as conversion of a N-acetyl group into an amino or amine group.

The capsular saccharide of serogroup C meningococcus is a homopolymer of sialic acid, linked α-2,9 with partial N-acetylation and sometimes O-acetylation at C-7/C-8:→9)-NeupNAc7/8OAc-(α2→, as shown in FIG. 7. Zwitterionization can be as for serogroup B.

The capsular saccharide of serogroup W135 meningococcus is a polymer of sialic acid-galactose disaccharide units [→4)-D-Neup5Ac(7/9OAc)-α-(2→6)-D-Gal-α-(1→], with variable O-acetylation at the 7 and 9 positions of the sialic acid (FIG. 8). The N. meningitidis serogroup Y saccharide is similar to the serogroup W135 saccharide, except that the disaccharide repeating unit includes glucose instead of galactose [→4)-D-Neup5Ac(7/9OAc)-α-(2→6)-D-Glc-α-(1→] (FIG. 9). The difference between W135 and Y is shown in FIG. 10, which shows that there is no difference in terms of charged groups, and so no difference in terms of zwitterionization. Every repeating unit includes a carboxyl group, which is anionic at physiological pH. A cationic amine or amino can be substituted for the N-acetyl group.

To avoid having the oppositely-charged groups on the same monomer in a homopolymers then it may be useful not to modify each wild-type repeating unit. For instance, in a homopolymer of -X-, where each X has a single anionic group, every third -X- could have a cationic group introduced. If one in every three -X- groups also has the anionic group removed and a cationic group introduced then, overall, the polymer has a repeating unit of -X₁-X₂-X₃- with an overall neutral charge.

Saccharides to be zwitterionized can be in their native form, or they may have been modified. For example, the saccharide may be shorter than the native capsular saccharide, or may be chemically modified.

Thus the saccharide may be a substantially full-length capsular polysaccharide, as found in nature, or it may be shorter than the natural length. Full-length polysaccharides may be depolymerized to give shorter fragments for use with the invention e.g. by hydrolysis in mild acid, by heating, by sizing chromatography, etc. Chain length has been reported to affect immunogenicity of GBS saccharides in rabbits [5], and the length of zwitterionic saccharides can influence their ability to stimulate T cells. For example, the B. fragilis saccharide has a native size of 129 kDa, retains its stimulatory activity when fragmented down as short as 17.1 kDa, but loses the activity when fragmented down to 5 kDa [6]. Zwitterionized saccharides are naturally processed to low molecular-weight carbohydrates by a NO-mediated mechanism [7], and any fragmentation should not prevent this processing.

Thus a zwitterionized saccharide of the invention preferably has a molecular weight of at least 6 kDa e.g. ≧7 kDa, ≧8 kDa, ≧9 kDa, ≧10 kDa, ≧12 kDa, ≧14 kDa, ≧15 kDa, ≧20 kDa, ≧25 kDa, ≧30 kDa, ≧35 kDa, ≧40 kDa, ≧45 kDa, ≧50 kDa, ≧75 kDa, ≧100 kDa, ≧125 kDa, etc.

Rather than express saccharide size by molecular weight, it is possible to express it in terms of the ‘degree of polymerization’ (DP) i.e. the number of repeating units in the saccharide. For a homopolymer, the degree of polymerisation is thus the same as the number of monosaccharide units. For a heteropolymer, however, the degree of polymerisation is the number of monosaccharide units in the whole chain divided by the number of monosaccharide units in the minimum repeating unit e.g. the DP of (Glc-Gal)₁₀ is 10 rather than 20, and the DP of (Glc-Gal-Neu)₁₀ is 10 rather than 30.

A zwitterionized saccharide of the invention preferably has a DP of at least 3 e.g. ≧4, ≧5, ≧6, ≧7, ≧8, ≧9, ≧10, ≧12, ≧14, ≧16, ≧18, ≧20, ≧25, ≧30, etc.

Depolymerisation of the serotype III capsular saccharide by endo-β-galactosidase has been reported [8-11]. Ozonolysis of capsular polysaccharides from GBS serotypes II, III and VIII has also been used for depolymerisation [12]. It is preferred to use saccharides with MW>30 kDa, and substantially full-length capsular polysaccharides can be used. For serotype Ia, it is preferred to use polysaccharides with a MW up to ˜145 kDa. For serotype Ib, it is preferred to use polysaccharides with a MW up to ˜50 kDa. For serotype III, it is preferred to use polysaccharides with a MW up to ˜50 kDa. These molecular masses can be measured by gel filtration relative to dextran standards, such as those available from Polymer Standard Service [13].

The saccharide may be chemically modified relative to the capsular saccharide as found in nature. For example, the saccharide may be de-O-acetylated (partially or fully), de-N-acetylated (partially or fully), N-propionated (partially or fully), etc. Depending on the particular saccharide, de-acetylation may or may not affect immunogenicity e.g. the NeisVac-C™ vaccine uses a de-O-acetylated saccharide, whereas Menjugate™ is acetylated, but both vaccines are effective. The relevance of O-acetylation on GBS saccharides in various serotypes is discussed in reference 14, and it is preferred to retain O-acetylation of sialic acid residues at positions 7, 8 and/or 9 before during and after purification e.g. by using formaldehyde for extraction of the saccharide and/or bacterial inactivation, by protection/de-protection, by re-acetylation, etc. The effect of de-acetylation etc. can be assessed by routine assays.

### Methods of Treatment and Administration of Saccharides

Saccharides of the invention are suitable as active ingredients in compositions for administration to human patients. Thus the invention provides a pharmaceutical composition comprising a saccharide of the invention. The composition is preferably an immunogenic composition, such as a vaccine, suitable for use in the prevention and/or treatment of a bacterial infection. Whereas the prior art conjugates these saccharides to carrier proteins, according to the invention the zwitterionized saccharides can be used without conjugation. Thus a saccharide of the invention is preferably not conjugated to a protein earner. In some embodiments, however, conjugation can be used.

These compositions will typically include pharmaceutically acceptable carriers. Such carriers include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. A thorough discussion of pharmaceutically acceptable excipients is available in reference 15.

Compositions may include an antimicrobial.

Compositions may comprise detergent e.g. a Tween (polysorbate), such as Tween 80. Detergents are generally present at low levels e.g. <0.01%.

The composition may be sterile.

Compositions of the invention are preferably non-pyrogenic e.g. containing <1 EU (endotoxin unit, a standard measure) per dose, and preferably <0.1 EU per dose.

Compositions of the invention are preferably gluten free.

Compositions will generally have an osmolality of between 200 mOsm/kg and 400 mOsm/kg, preferably between 240-360 mOsm/kg, and will more preferably fall within the range of 290-300 mOsm/kg. Compositions may be substantially isotonic with respect to humans.

Compositions may include sodium salts (e.g. sodium chloride) to give tonicity. A concentration of 10±2 mg/ml NaCl is typical.

Compositions of the invention may include one or more buffers. Typical buffers include: a phosphate buffer; a Tris buffer; a borate buffer; a succinate buffer; a histidine buffer; or a citrate buffer. A phosphate buffer is typical. Buffers will typically be included in the 5-20 mM range.

The pH of a composition of the invention will generally be between 5.0 and 7.5, and more typically between 5.0 and 6.0 for optimum stability, or between 6.0 and 7.0.

Compositions may comprise a sugar alcohol (e.g. mannitol) or a disaccharide (e.g. sucrose or trehalose) e.g. at around 15-30 mg/ml (e.g. 25 mg/ml), particularly if they are to be lyophilised or if they include material which has been reconstituted from lyophilised material.

The invention also provides a method of raising an immune response in a patient, comprising the step of administering a saccharide of the invention to the patient.

The invention also provides a saccharide of the invention for use in medicine.

The invention also provides the use of a saccharide of the invention in the manufacture of a medicament for administering to a patient.

Saccharides of the invention are preferably for the treatment of a human. Where a vaccine is for prophylactic use, the human is preferably a child (e.g. a toddler or infant) or a teenager; where the vaccine is for therapeutic use, the human is preferably an adult. A vaccine intended for children may also be administered to adults e.g. to assess safety, dosage, immunogenicity, etc. A preferred class of humans for treatment are females of child-bearing age (e.g. teenagers and above). Another preferred class is pregnant females. In order to have full efficacy, a typical immunization schedule for a child may involve administering more than one dose. For example, doses may be at: 0 & 6 months (time 0 being the first dose); at 0, 1, 2 & 6 months; at day 0, day 21 and then a third dose between 6 & 12 months; or at 0, 1, 2, 6 & 12 months.

Where using a modified GBS saccharide then the uses and methods of the invention are preferably for prevention and/or treatment of a disease caused by S. agalactiae e.g. neonatal sepsis or bacteremia, neonatal pneumonia, neonatal meningitis, endometritis, osteomyelitis, septic arthritis, etc.

When using a modified meningococcal saccharide then the uses and methods of the invention are preferably for prevention and/or treatment of a disease caused by N. meningitidis e.g. bacterial (or, more specifically, meningococcal) meningitis, or septicemia.

The subject in which disease is prevented may not be the same as the subject that receives the saccharide of the invention. For instance, a saccharide may be administered to a female (before or during pregnancy) in order to protect offspring (so-called ‘maternal immunisation’ [16-18]).

One way of checking efficacy of therapeutic treatment involves monitoring bacterial infection after administration of the composition of the invention. One way of checking efficacy of prophylactic treatment involves monitoring immune responses against the antigens after administration of the composition.

Preferred compositions of the invention can confer an antibody titre in a patient that is superior to the criterion for seroprotection for an acceptable percentage of human subjects (preferably for each antigenic component in compositions including multiple antigens). Antigens with an associated antibody titre above which a host is considered to be seroconverted against the antigen are well known, and such titres are published by organisations such as WHO. Preferably more than 80% of a statistically significant sample of subjects is seroconverted, more preferably more than 90%, still more preferably more than 93% and most preferably 96-100%.

Compositions of the invention can be administered by intramuscular injection e.g. into the arm or leg

Vaccines produced by the invention may be administered to patients at the same time as other immunogenic compositions.

Saccharides may be administered in conjunction with other immunoregulatory agents. In particular, compositions will usually include a vaccine adjuvant. Adjuvants which may be used in compositions of the invention include, but are not limited to:

### A. Mineral-Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, etc. [e.g. see chapters 8 & 9 of ref. 19], or mixtures of different mineral compounds, with the compounds taking any suitable form (e.g. gel, crystalline, amorphous, etc.), and with adsorption being preferred. The mineral containing compositions may also be formulated as a particle of metal salt [20].

Aluminium phosphates are particularly preferred, particularly in compositions which include a H. influenzae saccharide antigen, and a typical adjuvant is amorphous aluminium hydroxyphosphate with PO₄/Al molar ratio between 0.84 and 0.92, included at 0.6 mg Al³⁺/ml. Adsorption with a low dose of aluminium phosphate may be used e.g. between 50 and 100 μg Al³⁺ per saccharide per dose. Where there is more than one saccharide in a composition, not all of them need to be adsorbed.

### B. Oil Emulsions

Oil emulsion compositions suitable for use as adjuvants in the invention include squalene-water emulsions, such as MF59 [Chapter 10 of ref. 19; see also ref. 21] (5% Squalene, 0.5% Tween 80, and 0.5% Span 85, formulated into submicron particles using a microfluidizer). Complete Freund's adjuvant (CFA) and incomplete Freund's adjuvant (IFA) may also be used.

### C. Saponin Formulations [Chapter 22 of Ref. 19]

Saponin formulations may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 22. Saponin formulations may also comprise a sterol, such as cholesterol [23].

Combinations of saponins and cholesterols can be used to form unique particles called immunostimulating complexs (ISCOMs) [chapter 23 of ref. 19]. ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 23-25. Optionally, the ISCOMS may be devoid of additional detergent [26].

A review of the development of saponin based adjuvants can be found in refs. 27 & 28.

### D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in refs. 29-34. Virosomes are discussed further in, for example, ref. 35

### E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref. 36. Such “small particles” of 3dMPL are small enough to be sterile filtered through a 0.22 um membrane [36]. Other non-toxic LPS derivatives include monophosphoryl lipid A mimics, such as aminoalkyl glucosaminide phosphate derivatives e.g. RC-529 [37,38].

Lipid A derivatives include derivatives of lipid A from Escherichia coli such as OM-174. OM-174 is described for example in refs. 39 & 40.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 41, 42 and 43 disclose possible analog substitutions e.g. replacement of guanosine with 2′-deoxy-7-deazaguanosine. The adjuvant effect of CpG oligonucleotides is further discussed in refs. 44-49.

The CpG sequence may be directed to TLR9, such as the motif GTCGTT or TTCGTT [50]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 51-53. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 50 & 54-56.

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref. 57 and as parenteral adjuvants in ref. 58. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivatives thereof, particularly LT-K63 and LT-R72, as adjuvants can be found in refs. 59-66. Numerical reference for amino acid substitutions is preferably based on the alignments of the A and B subunits of ADP-ribosylating toxins set forth in ref. 67, specifically incorporated herein by reference in its entirety.

### F. Human Immunomodulators

Human immunomodulators suitable for use as adjuvants in the invention include cytokines, such as interleukins (e.g. IL-1, IL-2, IL-4, IL-5, IL-6, IL-7, IL-12 [68], etc.) [69], interferons (e.g. interferon-γ), macrophage colony stimulating factor, and tumor necrosis factor. A preferred immunomodulator is IL-12.

### G. Bioadhesives and Mucoadhesives

Bioadhesives and mucoadhesives may also be used as adjuvants in the invention. Suitable bioadhesives include esterified hyaluronic acid microspheres [70] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [71].

### H. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### I. Liposomes (Chapters 13 & 14 of Ref. 19)

Examples of liposome formulations suitable for use as adjuvants are described in refs. 72-74.

### J. Polyoxyethylene Ether and Polyoxyethylene Ester Formulations

Adjuvants suitable for use in the invention include polyoxyethylene ethers and polyoxyethylene esters [75]. Such formulations further include polyoxyethylene sorbitan ester surfactants in combination with an octoxynol [76] as well as polyoxyethylene alkyl ethers or ester surfactants in combination with at least one additional non-ionic surfactant such as an octoxynol [77]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### K. Polyphosphazene (PCPP)

PCPP formulations are described, for example, in refs. 78 and 79.

### L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

### M. Imidazoquinolone Compounds.

Examples of imidazoquinolone compounds suitable for use adjuvants in the invention include Imiquamod and its homologues (e.g. “Resiquimod 3M”), described further in refs. 80 and 81.

The invention may also comprise combinations of aspects of one or more of the adjuvants identified above. For example, the following adjuvant compositions may be used in the invention: (1) a saponin and an oil-in-water emulsion [82]; (2) a saponin (e.g. QS21)+a non-toxic LPS derivative (e.g. 3dMPL) [83]; (3) a saponin (e.g. QS21)+a non-toxic LPS derivative (e.g. 3dMPL)+a cholesterol; (4) a saponin (e.g. QS21)+3dMPL+IL-12 (optionally +a sterol) [84]; (5) combinations of 3dMPL with, for example, QS21 and/or oil-in-water emulsions [85]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (8) one or more mineral salts (such as an aluminum salt)+a non-toxic derivative of LPS (such as 3dMPL).

Other substances that act as immunostimulating agents are disclosed in chapter 7 of ref. 19.

The use of an aluminium hydroxide and/or aluminium phosphate adjuvant is particularly preferred, and antigens are generally adsorbed to these salts. Calcium phosphate is another preferred adjuvant.

Compositions of the invention will generally be in aqueous form.

### Combination Vaccines

Rather than using just a single modified saccharide of the invention, it is preferred to administer a composition comprising more than one saccharide. For example, a mixture of saccharides from 2, 3, 4 or 5 of meningococcal serogroups A, B, C, W135 and Y can be used. Similarly, a mixture of 2 or 3 of GBS serotypes Ia, Ib and III can be used.

For GBS, a combination of all three of serotypes Ia, Ib and III is preferred. The saccharide mixture may also be supplemented by one or more GBS protein antigens. Preferred protein antigens for use with the invention include those disclosed in references 86 and 87-89. Five preferred GBS protein antigens for use with the invention are known as: GBS67; GBS80; GBS104; GBS276; and GBS322 [see ref. 86]. Further details of these five antigens are given below.

The full-length sequences for these five GBS proteins are SEQ ID NOs 1 to 5 herein. Compositions of the invention may thus include (a) a polypeptide comprising an amino acid sequence selected from SEQ ID NOs 1 to 5, and/or (b) a polypeptide comprising (i) an amino acid sequence that has sequence identity to one or more of SEQ ID NOs 1 to 5 and/or (ii) a fragment of SEQ ID NOs 1 to 5.

Depending on the particular SEQ ID NO, the degree of sequence identity in (i) is preferably greater than 50% (e.g. 60%, 70%, 75%, 80%, 85%, 90%, 91%, 92%, 93%, 94%, 95%, 96%, 97%, 98%, 99% or more). These polypeptides include homologs, orthologs, allelic variants and functional mutants. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty=12 and gap extension penalty=1.

Depending on the particular SEQ ID NO, the fragments of (ii) should comprise at least n consecutive amino acids from the sequences and, depending on the particular sequence, n is 7 or more (e.g. 8, 10, 12, 14, 16, 18, 20, 22, 24, 26, 28, 30, 35, 40, 45, 50, 60, 70, 80, 90, 100 or more). The fragment may comprise at least one T-cell or, preferably, a B-cell epitope of the sequence. T- and B-cell epitopes can be identified empirically (e.g. using PEPSCAN [90,91] or similar methods), or they can be predicted (e.g. using the Jameson-Wolf antigenic index [92], matrix-based approaches [93], TEPITOPE [94], neural networks [95], OptiMer & EpiMer [96,97], ADEPT [98], Tsites [99], hydrophilicity [100], antigenic index [101] or the methods disclosed in reference 102 etc.). Other preferred fragments are SEQ ID NOs 1 to 5 without their N-terminal amino acid residue or without their N-terminal signal peptide. Removal of one or more domains, such as a leader or signal sequence region, a transmembrane region, a cytoplasmic region or a cell wall anchoring motif can be used. Preferred fragments are given below (SEQ ID NOs 6 to 19).

These polypeptide may, compared to SEQ ID NOs 1 to 5, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cystine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may also include one or more (e.g. 1, 2, 3, 4, 5, 6, 7, 8, 9, 10, etc.) single amino acid deletions relative to SEQ ID NOs 1 to 5. The polypeptides may also include one or more (e.g. 1, 2, 3, 4, 5, 6, 7, 8, 9, 10, etc.) insertions (e.g. each of 1, 2, 3, 4 or 5 amino acids) relative to the SEQ ID NOs 1 to 5.

Polypeptides of the invention can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides <40 amino acids long involves in vitro chemical synthesis [103,104]. Solid-phase peptide synthesis is particularly preferred, such as methods based on tBoc or Fmoc [105] chemistry. Enzymatic synthesis [106] may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [107]. Where D-amino acids are included, however, it is preferred to use chemical synthesis. Polypeptides of the invention may have covalent modifications at the C-terminus and/or N-terminus.

If these GBS proteins are included in compositions of the invention then they can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.). They are preferably used in purified or substantially purified form i.e. substantially free from other polypeptides (e.g. free from naturally-occurring polypeptides), particularly from other GBS or host cell polypeptides).

### GBS67

Nucleotide and amino acid sequence of GBS67 sequenced from serotype V strain 2603 V/R are set forth in ref. 86 as SEQ ID NOs 3745 & 3746. The amino acid sequence is SEQ ID NO:1 herein:

MRKYQKFSKILTLSLFCLSQIPLNTNVLGESTVPENGAKGKLVVKKTDDQNKPLSKATFVLKTTAHPESKIEKVTAELT
GEATFDNLIPGDYTLSEETAPEGYKKTNQTWQVKVESNGKTTIQNSGDKNSTIGQNQEELDKQYPPTGIYEDTKESYKL
EHVKGSVPNGKSEAKAVNPYSSEGEHIREIPEGTLSKRISEVGDLAHNKYKIELTVSGKTIVKPVDKQKPLDVVFVLDN
SNSMNNDGPNFQRHNKAKKAAEALGTAVKDILGANSDNRVALVTYGSDIFDGRSVDVVKGFKEDDKYYGLQTKFTIQTE
NYSHKQLTNNAEEIIKRIPTEAPKAKWGSTTNGLTPEQQKEYYLSKVGETFTMKAFMEADDILSQVNRNSQKIIVHVTD
GVPTRSYAINNFKLGASYESQFEQMKKNGYLNKSNFLLTDKPEDIKGNGESYFLFPLDSYQTQIISGNLQKLHYLDLNL
NYPKGTIYRNGPVKEHGTPTKLYINSLKQKNYDIFNFGIDISGFRQVYNEEYKKNQDGTFQKLKEEAFKLSDGEITELM
RSFSSKPEYYTPIVTSADTSNNEILSKIQQQFETILTKENSIVNGTIEDPMGDKINLQLGNGQTLQPSDYTLQGNDGSV
MKDGIATGGPNNDGGILKGVKLEYIGNKLYVRGLNLGEGQKVTLTYDVKLDDSFISNKFYDTNGRTTLNPKSEDPNTLR
DFPIPKIRDVREYPTITIKNEKKLGEIEFIKVDKDNNKLLLKGATFELQEFNEDYKLYLPIKNNNSKVVTGENGKISYK
DLKDGKYQLIEAVSPEDYQKITNKPILTFEVVKGSIKNIIAVNKQISEYHEEGDKHLITNTHIPPKGIIPMTGGKGILS
FILIGGAMMSIAGGIYIWKRYKKSSDMSIKKD

GBS67 contains a C-terminus transmembrane region which is indicated by the underlined region closest to the C-terminus of SEQ ID NO: 1 above. One or more amino acids from the transmembrane region may be removed, or the amino acid may be truncated before the transmembrane region. An example of such a GBS67 fragment is set forth below as SEQ ID NO: 18.

MRKYQKFSKILTLSLFCLSQIPLNTNVLGESTVPENGAKGKLVVKKTDDQNKPLSKATFVLKTTAHPESKIEKVTAELT
GEATFDNLIPGDYTLSEETAPEGYKKTNQTWQVKVESNGKTTIQNSGDKNSTIGQNQEELDKQYPPTGIYEDTKESYKL
EHVKGSVPNGKSEAKAVNPYSSEGEHIREIPEGTLSKRISEVGDLAHNKYKIELTVSGKTIVKPVDKQKPLDVVFVLDN
SNSMNNDGPNFQRHNKAKKAAEALGTAVKDILGANSDNRVALVTYGSDIFDGRSVDVVKGFKEDDKYYGLQTKFTIQTE
NYSHKQLTNNAEEIIKRIPTEAPKAKWGSTTNGLTPEQQKEYYLSKVGETFTMKAFMEADDILSQVNRNSQKIIVHVTD
GVPTRSYAINNFKLGASYESQFEQMKKNGYLNKSNFLLTDKPEDIKGNGESYFLFPLDSYQTQIISGNLQKLHYLDLNL
NYPKGTIYRNGPVKEHGTPTKLYINSLKQKNYDIFNFGIDISGFRQVYNEEYKKNQDGTFQKLKEEAFKLSDGEITELM
RSFSSKPEYYTPIVTSADTSNNEILSKIQQQFETILTKENSIVNGTIEDPMGDKINLQLGNGQTLQPSDYTLQGNDGSV
MKDGIATGGPNNDGGILKGVKLEYIGNKLYVRGLNLGEGQKVTLTYDVKLDDSFISNKFYDTNGRTTLNPKSEDPNTLR
DFPIPKIRDVREYPTITIKNEKKLGEIEFIKVDKDNNKLLLKGATFELQEFNEDYKLYLPIKNNNSKVVTGENGKISYK
DLKDGKYQLIEAVSPEDYQKITNKPILTFEVVKGSIKNIIAVNKQISEYHEEGDKHLITNTHIPPKGIIPMTGGKGILS

GBS67 contains an amino acid motif indicative of a cell wall anchor, shown in italics in SEQ ID NO: 1 above. In some recombinant host cell systems, it may be preferable to remove this motif to facilitate secretion of a recombinant GBS67 protein from the host cell. Accordingly, in one preferred fragment of GBS67 for use in the invention, the transmembrane and the cell wall anchor motif are removed from GBS67. An example of such a GBS67 fragment is set forth below as SEQ ID NO: 19.

MRKYQKFSKILTLSLFCLSQIPLNTNVLGESTVPENGAKGKLVVKKTDDQNKPLSKATFVLKTTAHPESKIEKVTAELT
GEATFDNLIPGDYTLSEETAPEGYKKTNQTWQVKVESNGKTTIQNSGDKNSTIGQNQEELDKQYPPTGIYEDTKESYKL
EHVKGSVPNGKSEAKAVNPYSSEGEHIREIPEGTLSKRISEVGDLAHNKYKIELTVSGKTIVKPVDKQKPLDVVFVLDN
SNSMNNDGPNFQRHNKAKKAAEALGTAVKDILGANSDNRVALVTYGSDIFDGRSVDVVKGFKEDDKYYGLQTKFTIQTE
NYSHKQLTNNAEEIIKRIPTEAPKAKWGSTTNGLTPEQQKEYYLSKVGETFTMKAFMEADDILSQVNRNSQKIIVHVTD
GVPTRSYAINNFKLGASYESQFEQMKKNGYLNKSNFLLTDKPEDIKGNGESYFLFPLDSYQTQIISGNLQKLHYLDLNL
NYPKGTIYRNGPVKEHGTPTKLYINSLKQKNYDIFNFGIDISGFRQVYNEEYKKNQDGTFQKLKEEAFKLSDGEITELM
RSFSSKPEYYTPIVTSADTSNNEILSKIQQQFETILTKENSIVNGTIEDPMGDKINLQLGNGQTLQPSDYTLQGNDGSV
MKDGIATGGPNNDGGILKGVKLEYIGNKLYVRGLNLGEGQKVTLTYDVKLDDSFISNKFYDTNGRTTLNPKSEDPNTLR
DFPIPKIRDVREYPTITIKNEKKLGEIEFIKVDKDNNKLLLKGATFELQEFNEDYKLYLPIKNNNSKVVTGENGKISYK
DLKDGKYQLIEAVSPEDYQKITNKPILTFEVVKGSIKNITAVNKQISEYHEEGDKHLITNTHIPPKGI

### GBS80

GBS80 refers to a putative cell wall surface anchor family protein. Nucleotide and amino acid sequence of GBS80 sequenced from serotype V isolated strain 2603 V/R are set forth in ref. 86 as SEQ ID NOs 8779 & 8780. The amino acid sequence is set forth below as SEQ ID NO: 2:

MKLSKKLLFSAAVLTMVAGSTVEPVAQFATGMSIVRAAEVSQERPAKTTVNIYKLQADSYKSEITSNGGIENKDGEVIS
NYAKLGDNVKGLQGVQFKRYKVKTDISVDELKKLTTVEAADAKVGTILEEGVSLPQKTNAQGLVVDALDSKSNVRYLYV
EDLKNSPSNITKAYAVPFVLELPVANSTGTGFLSEINIYPKNVVTDEPKTDKDVKKLGQDDAGYTIGEEFKWFLKSTIP
ANLGDYEKFEITDKFADGLTYKSVGKIKIGSKTLNRDEHYTIDEPTVDNQNTLKITFKPEKFKEIAELLKGMTLVKNQD
ALDKATANTDDAAFLEIPVASTINEKAVLGKAIENTFELQYDHTPDKADNPKPSNPPRKPEVHTGGKRFVKKDSTETQT
LGGAEFDLLASDGTAVKWTDALIKANTNKNYIAGEAVTGQPIKLKSHTDGTFEIKGLAYAVDANAEGTAVTYKLKETKA
PEGYVIPDKEIEFTVSQTSYNTKPTDITVDSADATPDTIKNNKRPSIPNTGGIGTAIFVAIGAAVMAFAVKGMKRRTKD
N

GBS80 contains a N-terminal leader or signal sequence region which is indicated by the underlined sequence above. One or more amino acids from the leader or signal sequence region of GBS80 can be removed. An example of such a GBS80 fragment is set forth below as SEQ ID NO: 6:

AEVSQERPAKTTVNIYKLQADSYKSEITSNGGIENKDGEVISNYAKLGDN
VKGLQGVQFKRYKVKTDISVDELKKLTTVEAADAKVGTILEEGVSLPQKT
NAQGLVVDALDSKSNVRYLYVEDLKNSPSNITKAYAVPFVLELPVANSTG
TGFLSEINIYPKNVVTDEPKTDKDVKKLGQDDAGYTIGEEFKWFLKSTIP
ANLGDYEKFEITDKFADGLTYKSVGKIKIGSKTLNRDEHYTIDEPTVDNQ
NTLKITFKPEKFKEIAELLKGMTLVKNQDALDKATANTDDAAFLEIPVAS
TINEKAVLGKAIENTFELQYDHTPDKADNPKPSNPPRKPEVHTGGKRFVK
KDSTETQTLGGAEFDLLASDGTAVKWTDALIKANTNKNYIAGEAVTGQPI
KLKSHTDGTFEIKGLAYAVDANAEGTAVTYKLKETKAPEGYVIPDKEIEF
TVSQTSYNTKPTDITVDSADATPDTIKNNKRPSIPNTGGIGTAIFVAIGA
AVMAFAVKGMKRRTKDN

GBS80 contains a C-terminal transmembrane region which is indicated by the underlined sequence near the end of SEQ ID NO: 2 above. One or more amino acids from the transmembrane region and/or a cytoplasmic region may be removed. An example of such a fragment is set forth below as SEQ ID NO:7:

MKLSKKLLFSAAVLTMVAGSTVEPVAQFATGMSIVRAAEVSQERPAKTTV
NIYKLQADSYKSEITSNGGIENKDGEVISNYAKLGDNVKGLQGVQFKRYK
VKTDISVDELKKLTTVEAADAKVGTILEEGVSLPQKTNAQGLVVDALDSK
SNVRYLYVEDLKNSPSNITKAYAVPFVLELPVANSTGTGFLSEINIYPKN
VVTDEPKTDKDVKKLGQDDAGYTIGEEFKWFLKSTIPANLGDYEKFEITD
KFADGLTYKSVGKIKIGSKTLNRDEHYTIDEPTVDNQNTLKITFKPEKFK
EIAELLKGMTLVKNQDALDKATANTDDAAFLEIPVASTINEKAVLGKAIE
NTFELQYDHTPDKADNPKPSNPPRKPEVHTGGKRFVKKDSTETQTLGGAE
FDLLASDGTAVKWTDALIKANTNKNYIAGEAVTGQPIKLKSHTDGTFEIK
GLAYAVDANAEGTAVTYKLKETKAPEGYVIPDKEIEFTVSQTSYNTKPTD
ITVDSADATPDTIKNNKRPSIPNTG

GBS80 contains an amino acid motif indicative of a cell wall anchor, shown in italics in SEQ ID NO: 2 above. In some recombinant host cell systems, it may be preferable to remove this motif to facilitate secretion of a recombinant GBS80 protein from the host cell. Thus the transmembrane and/or cytoplasmic regions and the cell wall anchor motif may be removed from GBS80. An example of such a fragment is set forth below as SEQ ID NO: 8.

MKLSKKLLFSAAVLTMVAGSTVEPVAQFATGMSIVRAAEVSQERPAKTTV
NIYKLQADSYKSEITSNGGIENKDGEVISNYAKLGDNVKGLQGVQFKRYK
VKTDISVDELKKLTTVEAADAKVGTILEEGVSLPQKTNAQGLVVDALDSK
SNVRYLYVEDLKNSPSNITKAYAVPFVLELPVANSTGTGFLSEINIYPKN
VVTDEPKTDKDVKKLGQDDAGYTIGEEFKWFLKSTIPANLGDYEKFEITD
KFADGLTYKSVGKIKIGSKTLNRDEHYTIDEPTVDNQNTLKITFKPEKFK
EIAELLKGMTLVKNQDALDKATANTDDAAFLEIPVASTINEKAVLGKAIE
NTFELQYDHTPDKADNPKPSNPPRKPEVHTGGKRFVKKDSTETQTLGGAE
FDLLASDGTAVKWTDALIKANTNKNYIAGEAVTGQPIKLKSHTDGTFEIK
GLAYAVDANAEGTAVTYKLKETKAPEGYVIPDKEIEFTVSQTSYNTKPTD
TTVDSADATPDTIKNNKRPS

Alternatively, in some recombinant host cell systems, it may be preferable to use the cell wall anchor motif to anchor the recombinantly expressed protein to the cell wall. The extracellular domain of the expressed protein may be cleaved during purification or the recombinant protein may be left attached to either inactivated host cells or cell membranes in the final composition.

In one embodiment, the leader or signal sequence region, the transmembrane and cytoplasmic regions and the cell wall anchor motif are removed from the GBS80 sequence. An example of such a GBS80 fragment is set forth below as SEQ ID NO: 9:

AEVSQERPAKTTVNIYKLQADSYKSEITSNGGIENKDGEVISNYAKLGDN
VKGLQGVQFKRYKVKTDISVDELKKLTTVEAADAKVGTILEEGVSLPQKT
NAQGLVVDALDSKSNVRYLYVEDLKNSPSNITKAYAVPFVLELPVANSTG
TGFLSEINIYPKNVVTDEPKTDKDVKKLGQDDAGYTIGEEFKWFLKSTIP
ANLGDYEKFEITDKFADGLTYKSVGKIKIGSKTLNRDEHYTIDEPTVDNQ
NTLKITFKPEKFKEIAELLKGMTLVKNQDALDKATANTDDAAFLEIPVAS
TINEKAVLGKAIENTFELQYDHTPDKADNPKPSNPPRKPEVHTGGKRFVK
KDSTETQTLGGAEFDLLASDGTAVKWTDALIKANTNKNYIAGEAVTGQPI
KLKSHTDGTFEIKGLAYAVDANAEGTAVTYKLKETKAPEGYVIPDKEIEF
TVSQTSYNTKPTDITVDSADATPDTIKNNKRPS

A particularly immunogenic fragment of GBS80 is located towards the N-terminus of the protein, and is given herein as SEQ ID NO: 10:

AEVSQERPAKTTVNIYKLQADSYKSEITSNGGIENKDGEVISNYAKLGDN
VKGLQGVQFKRYKVKTDTSVDELKKLTTVEAADAKVGTILEEGVSLPQKT
NAQGLVVDALDSKSNVRYLYVEDLKNSPSNITKAYAVPFVLELPVANSTG
TGFLSEINIYPKNVVTDEPKTDKDVKKLGQDDAGYTTGEEFKWFLKSTIP
ANLGDYEKFEITDKFADGLTYKSVGKIKIGSKTLNRDEHYTIDEPTVDNQ
NTLKITFKPEKFKEIAELLKG

### GBS104

GBS104 refers to a putative cell wall surface anchor family protein. It has been referred to as emaA.

Nucleotide and amino acid sequences of GBS104 sequenced from serotype V isolated strain 2603 V/R are set forth in Ref. 86 as SEQ ID 8777 and SEQ ID 8778. The amino acid sequence is SEQ ID NO: 3 herein:

MKKRQKIWRGLSVTLLILSQIPFGILVQGETQDTNQALGKVIVKKTGDNA
TPLGKATFVLKNDNDKSETSHETVEGSGEATFENIKPGDYTLREETAPIG
YKKTDKTWKVKVADNGATIIEGMDADKAEKRKEVLNAQYPKSAIYEDTKE
NYPLVNVEGSKVGEQYKALNPINGKDGRREIAEGWLSKKITGVNDLDKNK
YKIELTVEGKTTVETKELNQPLDVVVLLDNSNSMNNERANNSQRALKAGE
AVEKLIDKITSNKDNRVALVTYASTIFDGTEATVSKGVADQNGKALNDSV
SWDYHKTTFTATTHNYSYLNLTNDANEVNILKSRIPKEAEHINGDRTLYQ
FGATFTQKALMKANEILETQSSNARKKLIFHVTDGVPTMSYAINFNPYIS
TSYQNQFNSFLNKIPDRSGILQEDFIINGDDYQIVKGDGESFKLFSDRKV
PVTGGTTQAAYRVPQNQLSVMSNEGYAINSGYIYLYWRDYNWVYPFDPKT
KKVSATKQIKTHGEPTTLYFNGNIRPKGYDIFTVGIGVNGDPGATPLEAE
KFMQSISSKTENYTNVDDTNKIYDELNKYFKTIVEEKHSIVDGNVTDPMG
EMIEFQLKNGQSFTHDDYVLVGNDGSQLKNGVALGGPNSDGGILKDVTVT
YDKTSQTIKINHLNLGSGQKVVLTYDVRLKDNYISNKFYNTNNRTTLSPK
SEKEPNTIRDFPIPKIRDVREFPVLTISNQKKMGEVEFINKVNKDHSESL
LGAKFQLQIEKDFSGYKQFVPEGSDVTTKNDGKIYFKALQDGNYKLYEIS
SPDGYIEVKTKPVVTFTIQNGEVTNLKADPNANKNQIGYLEGNGKHLITN
TPKREPGVFPKTGGIGTIVYILVGSTFMILTICSFRRKQL

GBS104 contains an N-terminal leader or signal sequence region which is indicated by the underlined sequence at the beginning of SEQ ID NO: 3 above. One or more amino acids from the leader or signal sequence region of GBS104 may be removed. An example of such a GBS104 fragment is set forth below as SEQ ID NO 11.

GETQDTNQALGKVIVKKTGDNATPLGKATFVLKNDNDKSETSHETVEGSG
EATFENIKPGDYTLREETAPIGYKKTDKTWKVKVADNGATIIEGMDADKA
EKRKEVLNAQYPKSAIYEDTKENYPLVNVEGSKVGEQYKALNPINGKDGR
REIAEGWLSKKITGVNDLDKNKYKIELTVEGKTTVETKELNQPLDVVVLL
DNSNSMNNERANNSQRALKAGEAVEKLIDKITSNKDNRVALVTYASTIFD
GTEATVSKGVADQNGKALNDSVSWDYHKTTFTATTHNYSYLNLTNDANEV
NILKSRIPKEAEHINGDRTLYQFGATFTQKALMKANEILETQSSNARKKL
IFHVTDGVPTMSYAINFNPYISTSYQNQFNSFLNKIPDRSGILQEDFIIN
GDDYQIVKGDGESFKLFSDRKVPVTGGTTQAAYRVPQNQLSVMSNEGYAI
NSGYIYLYWRDYNWVYPFDPKTKKVSATKQIKTHGEPTTLYFNGNIRPKG
YDIFTVGIGVNGDPGATPLEAEKFMQSISSKTENYTNVDDTNKIYDELNK
YFKTIVEEKHSIVDGNVTDPMGEMIEFQLKNGQSFTHDDYVLVGNDGSQL
KNGVALGGPNSDGGILKDVTVTYDKTSQTIKINHLNLGSGQKVVLTYDVR
LKDNYISNKFYNTNNRTTLSPKSEKEPNTIRDFPIPKIRDVREFPVLTIS
NQKKMGEVEFIKVNKDKHSESLLGAKFQLQIEKDFSGYKQFVPEGSDVTT
KNDGKIYFKALQDGNYKLYEISSPDGYIEVKTKPVVTFTIQNGEVTNKLA
DPNANKNQIGYLEGNGKHLITNTPKRPPGVFPKTGGIGTIVYILVGSTFM
ILTICSFRRKQL

GBS104 contains a C-terminal transmembrane and/or cytoplasmic region which is indicated by the underlined region near the end of SEQ ID NO:3 above. One or more amino acids from the transmembrane or cytoplasmic regions may be removed. An example of such a GBS104 fragment is set forth below as SEQ ID NO 12:

MKKRQKIWRGLSVTLLILSQIPFGILVQGETQDTNQALGKVIVKKTGDNA
TPLGKATFVLKNDNDKSETSHETVEGSGEATFENIKPGDYTLREETAPIG
YKKTDKTWKVKVADNGATIIEGMDADKAEKRKEVLNAQYPKSAIYEDTKE
NYPLVNVEGSKVGEQYKALNPINGKDGRREIAEGWLSKKITGVNDLDKNK
YKIELTVEGKTTVETKELNQPLDVVVLLDNSNSMNNERANNSQRALKAGE
AVEKLIDKITSNKDNRVALVTYASTIFDGTEATVSKGVADQNGKALNDSV
SWDYHKTTFTATTHNYSYLNLTNDANEVNILKSRIPKEAEHINGDRTLYQ
FGATFTQKALMKANEILETQSSNARKKLIFHVTDGVPTMSYAINFNPYIS
TSYQNQFNSFLNKIPDRSGILQEDFIINGDDYQIVKGDGESFKLFSDRKV
PVTGGTTQAAYRVPQNQLSVMSNEGYAINSGYIYLYWRDYNWVYPFDPKT
KKVSATKQIKTHGEPTTLYFNGNIRPKGYDIFTVGIGVNGDPGATPLEAE
KFMQSISSKTENYTNVDDTNKIYDELNKYFKTIVEEKHSIVDGNVTDPMG
EMIEFQLKNGQSFTHDDYVLVGNDGSQLKNGVALGGPNSDGGILKDVTVT
YDKTSQTIKINHLNLGSGQKVVLTYDVRLKDNYISNKFYNTNNRTTLSPK
SEKEPNTIRDFPIPKIRDVREFPVLTISNQKKMGEVEFIKVNKDKHSESL
LGAKFQLQIEKDFSGYKQFVPEGSDVTTKNDGKIYFKALQDGNYKLYEIS
SPDGYIEVKTKPVVTFTIQNGEVTNLKADPNANKNQIGYLEGNGKHLITN
T

One or more amino acids from the leader or signal sequence region and one or more amino acids from the transmembrane or cytoplasmic regions may be removed. An example of such a GBS104 fragment is set forth below as SEQ ID NO 13:

GETQDTNQALGKVIVKKTGDNATPLGKATFVLKNDNDKSETSHETVEGSG
EATFENIKPGDYTLREETAPIGYKKTDKTWKVKVADNGATIIEGMDADKA
EKRKEVLNAQYPKSAIYEDTKENYPLVNVEGSKVGEQYKALNPINGKDGR
REIAEGWLSKKITGVNDLDKNKYKIELTVEGKTTVETKELNQPLDVVVLL
DNSNSMNNERANNSQRALKAGEAVEKLIDKITSNKDNRVALVTYASTIFD
GTEATVSKGVADQNGKALNDSVSWDYHKTTFTATTHNYSYLNLTNDANEV
NILKSRIPKEAEHINGDRTLYQFGATFTQKALMKANEILETQSSNARKKL
IFHVTDGVPTMSYAINFNPYISTSYQNQFNSFLNKIPDRSGILQEDFIIN
GDDYQIVKGDGESFKLFSDRKVPVTGGTTQAAYRVPQNQLSVMSNEGYAI
NSGYIYLYWRDYNWVYPFDPKTKKVSATKQIKTHGEPTTLYFNGNIRPKG
YDIFTVGIGVNGDPGATPLEAEKFMQSISSKTENYTNVDDTNKIYDELNK
YFKTIVEEKHSIVDGNVTDPMGEMIEFQLKNGQSFTHDDYVLVGNDGSQL
KNGVALGGPNSDGGILKDVTVTYDKTSQTIKINHLNLGSGQKVVLTYDVR
LKDNYISNKFYNTNNRTTLSPKSEKEPNTIRDFPIPKIRDVREFPVLTIS
NQKKMGEVEFIKVNKDKHSESLLGAKFQLQIEKDFSGYKQFVPEGSDVTT
KNDGKIYFKALQDGNYKLYEISSPDGYIEVKTKPVVTFTIQNGEVTNLKA
DPNANKNQIGYLEGNGKHLITNT

Further fragments of GBS104 include an 830 amino acid fragment of GBS104 of amino acids 28-858 (numbered by SEQ ID NO: 3), a 359 amino acid fragment of GBS104 of amino acids 28-387, a 581 amino acid fragment of GBS104 of amino acids 28-609, or a 740 amino acid fragment of GBS104 of amino acids 28-768.

### GBS276

GBS276 refers to a C5a peptidase. Further description of GBS276 can be found in references 108-111. Nucleotide and amino acid sequences of GBS276 sequenced from serotype V isolated strain 2603 V/R are set forth in Ref. 86 as SEQ ID NOs 8941 & 8942. The amino acid sequence is SEQ ID NO: 4 herein:

MRKKQKLPFDKLAIALISTSILLNAQSDIKANTVTEDTPATEQAVEPPQP
IAVSEESRSSKETKTSQTPSDVGETVADDANDLAPQAPAKTADTPATSKA
TIRDLNDPSHVKTLQEKAGKGAGTVVAVIDAGFDKNHEAWRLTDKTKARY
QSKENLEKAKKEHGITYGEWVNDKVAYYHDYSKDGKNAVDQEHGTHVSGI
LSGNAPSEMKEPYRLEGAMPEAQLLLMRVEIVNGLADYARNYAQAIRDAV
NLGAKVINMSFGNAALAYANLPDETKKAFDYAKSKGVSIVTSAGNDSSFG
GKPRLPLADHPDYGVVGTPAAADSTLTVASYSPDKQLTETATVKTDDHQD
KEMPVISTNRFEPNKAYDYAYANRGTKEDDFKDVEGKIALIERGDIDFKD
KIANAKKAGAVGVLIYDNQDKGFPIELPNVDQMPAAFISRRDGLLLKDNP
PKTITFNATPKVLPTASGTKLSRFSSWGLTADGNIKPDIAAPGQDILSSV
ANNKYAKLSGTSMSAPLVAGIMGLLQKQYETQYPDMTPSERLDLAKKVLM
SSATALYDEDEKAYFSPRQQGAGAVDAKKASAATMYVTDKDNTSSKVHLN
NVSDKFEVTVTVHNKSDKPQELYYQVTVQTDKVDGKHFALAPKALYETSW
QKITIPANSSKQVTVPIDASRFSKDLLAQMKNGYFLEGFVRFKQDPTKEE
LMSIPYIGFRGDFGNLSALEKPIYDSKDGSSYYHEANSDAKDQLDGDGLQ
FYALKNNFTALTTESNPWTIIKAVKEGVENIEDIESSEITETIFAGTFAK
QDDDSHYYIHRHANGKPYAAISPNGDGNRDYVQFQGTFLRNAKNLVAEVL
DKEGNVVWTSEVTEQVVKNYNNDLASTLGSTRFEKTRWDGKDKDGKVVAN
GTYTYRVRYTPISSGAKEQHTDFDVIVDNTTPEVATSATFSTEDSRLTLA
SKPKTSQPVYRERIAYTYMDEDLPTTEYISPNEDGTFTLPEEAETMEGAT
VPLKMSDFTYVVEDMAGNITYTPVTKLLEGHSNKPEQDGSDQAPDKKPEA
KPEQDGSGQTPDKKKETKPEKDSSGQTPGKTPQKGQSSRTLEKRSSKRAL
ATKASTRDQLPTTNDKDTNRLHLLKLVMTTFFLG

GBS276 contains an N-terminal leader or signal sequence region which is indicated by the underlined sequence at the beginning of SEQ ID NO: 4 above. One or more amino acids from the leader or signal sequence region of GBS276 may be removed. An example of such a GBS276 fragment is set forth below as SEQ ID NO: 14:

QSDIKANTVTEDTPATEQAVEPPQPIAVSEESRSSKETKTSQTPSDVGET
VADDANDLAPQAPAKTADTPATSKATIRDLNDPSHVKTLQEKAGKGAGTV
VAVIDAGFDKNHEAWRLTDKTKARYQSKENLEKAKKEHGITYGEWVNDKV
AYYHDYSKDGKNAVDQEHGTHVSGILSGNAPSEMKEPYRLEGAMPEAQLL
LMRVEIVNGLADYARNYAQAIRDAVNLGAKVINMSFGNAALAYANLPDET
KKAFDYAKSKGVSIVTSAGNDSSFGGKPRLPLADHPDYGVVGTPAAADST
LTVASYSPDKQLTETATVKTDDHQDKEMPVISTNRFEPNKAYDYAYANRG
TKEDDFKDVEGKIALIERGDIDFKDKIANAKKAGAVGVLIYDNQDKGFPI
ELPNVDQMPAAFISRRDGLLLKDNPPKTITFNATPKVLPTASGTKLSRFS
SWGLTADGNIKPDIAAPGQDILSSVANNKYAKLSGTSMSAPLVAGIMGLL
QKQYETQYPDMTPSERLDLAKKVLMSSATALYDEDEKAYFSPRQQGAGAV
DAKKASAATMYVTDKDNTSSKVHLNNVSDKFEVTVTVHNKSDKPQELYYQ
VTVQTDKVDGKHFALAPKALYETSWQKITIPANSSKQVTVPIDASRFSKD
LLAQMKNGYFLSGFVRFKQDPTKEELMSIPYIGFRGDFGNLSALEKPIYD
SKDGSSYYHEANSDAKDQLDGDGLQFYALKNNFTALTTESNPWTIIKAVK
EGVENIEDIESSEITETIFAGTFAKQDDDSHYYIHRHANGKPYAAISPNG
DGNRDYVQFQGTFLRNAKNLVAEVLDKEGNVVWTSEVTEQVVKNYNNDLA
STLGSTRFEKTRWDGKDKDGKVVANGTYTYRVRYTPISSGAKEQHTDFDV
IVDNTTPEVATSATFSTEDSRLTLASKPKTSQPVYRERIAYTYMDEDLPT
TEYISPNEDGTFTLPEEAETMEGATVPLKMSDFTYVVEDMAGNITYTPVT
KLLEGHSNKPEQDGSDQAPDKKPEAKPEQDGSGQTPDKKKETKPEKDSSG
QTPGKTPQKGQSSRTLEKRSSKRALATKASTRDQLPTTNDKDTNRLHLLK
LVMTTFFLG

GBS276 contains a C-terminal transmembrane and/or cytoplasmic region which is indicated by the underlined sequence near the end of SEQ ID NO: 4 above. One or more amino acids from the transmembrane or cytoplasmic regions of GBS276 may be removed. An example of such a GBS276 fragment is set forth below as SEQ ID NO: 15:

MRKKQKLPFDKLAIALISTSILLNAQSDIKANTVTEDTPATEQAVEPPQP
IAVSEESRSSKETKTSQTPSDVGETVADDANDLAPQAPAKTADTPATSKA
TIRDLNDPSHVKTLQEKAGKGAGTVVAVIDAGFDKNHEAWRLTDKTKARY
QSKENLEKAKKEHGITYGEWVNDKVAYYHDYSKDGKNAVDQEBGTHVSGI
LSGNAPSEMKEPYRLEGAMPEAQLLLMRVEIVNGLADYARNYAQAIRDAV
NLGAKVINMSFGNAALAYANLPDETKKAFDYAKSKGVSIVTSAGNDSSFG
GKPRLPLADHPDYGVVGTPAAADSTLTVASYSPDKQLTETATVKTDDHQD
KEMPVISTNRFEPNKAYDYAYANRGTKEDDFKDVEGKIALIERGDIDFKD
KIANAKKAGAVGVLIYDNQDKGFPIELPNVDQMPAAFISRRDGLLLKDNP
PKTITFNATPKVLPTASGTKLSRFSSWGLTADGNIKPDIAAPGQDILSSV
ANNKYAKLSGTSMSAPLVAGIMGLLQKQYETQYPDMTPSERLDLAKKVLM
SSATALYDEDEKAYFSPRQQGAGAVDAKKASAATMYVTDKDNTSSKVHLN
NVSDKFEVTVTVHNKSDKPQELYYQVTVQTDKVDGKHFALAPKALYETSW
QKITIPANSSKQVTVPIDASRFSKDLLAQMKNGYFLEGFVRFKQDPTKEE
LMSIPYIGFRGDFGNLSALEKPIYDSKDGSSYYHEANSDAKDQLDGDGLQ
FYALKNNFTALTTESNPWTIIKAVKEGVENIEDIESSEITETIFAGTFAK
QDDDSHYYIHRHANGKPYAAISPNGDGNRDYVQFQGTFLRNAKNLVAEVL
DKEGNVVWTSEVTEQVVKNYNNDLASTLGSTRFEKTRWDGKDKDGKVVAN
GTYTYRVRYTPISSGAKEQHTDFDVIVDNTTPEVATSATFSTEDSRLTLA
SKPKTSQPVYRERIAYTYMDEDLPTTEYISPNEDGTFTLPEEAETMEGAT
VPLKMSDFTYVVEDMAGNITYTPVTKLLEGHSNKPEQDGSDQAPDKKPEA
KPEQDGSGQTPDKKKETKPEKDSSGQTPGKTPQKGQSSRTLEKRSSKRAL
ATK

One or more amino acids from the leader or signal sequence region and one or more amino acids from the transmembrane or cytoplasmic regions of GBS276 may be removed. An example of such a GBS276 fragment is set forth below as SEQ ID NO: 16:

QSDIKANTVTEDTPATEQAVEPPQPIAVSEESRSSKETKTSQTPSDVGET
VADDANDLAPQAPAKTADTPATSKATIRDLNDPSHVKTLQFKAGKGAGTV
VAVIDAGFDKNHEAWRLTDKTKARYQSKENLEKAKKEHGITYGEWVNDKV
AYYHDYSKDGKNAVDQEHGTHVSGILSGNAPSEMKEPYRLEGAMPEAQLL
LMRVEIVNGLADYARNYAQAIRDAVNLGAKVINMSFGNAALAYANLPDET
KKAFDYAKSKGVSIVTSAGNDSSFGGKPRLPLADHPDYGVVGTPAAADST
LTVASYSPDKQLTETATVKTDDHQDKEMPVISTNRFEPNKAYDYAYANRG
TKEDDFKDVEGKIALIERGDIDFKDKIANAKKAGAVGVLIYDNQDKGFPI
ELPNVDQMPAAFISRRDGLLLKDNPPKTITFNATPKVLPTASGTKLSRFS
SWGLTADGNIKPDIAAPGQDILSSVANNKYAKLSGTSMSAPLVAGIMGLL
QKQYETQYPDMTPSERLDLAKKVLMSSATALYDEDEKAYFSPRQQGAGAV
DAKKASAATMYVTDKDNTSSKVHLNNVSDKFEVTVTVHNKSDKPQELYYQ
VTVQTDKVDGKHFALAPKALYETSWQKITIPANSSKQVTVPIDASRFSKD
LLAQMKNGYFLEGFVRFKQDPTKEELMSIPYIGFRGDFGNLSALEKPIYD
SKDGSSYYHEANSDAKDQLDGDGLQFYALKNNFTALTTESNPWTIIKAVK
EGVENIEDIESSEITETIFAGTFAKQDDDSHYYIHRHANGKPYAAISPNG
DGNRDYVQFQGTFLRNAKNLVAEVLDKEGNVVWTSEVTEQVVKNYNNDLA
STLGSTRFEKTRWDGKDKDGKVVANGTYTYRVRYTPISSGAKEQHTDFDV
IVDNTTPEVATSATFSTEDSRLTLASKPKTSQPVYRERIAYTYMDEDLPT
TEYISPNEDGTFTLPEEAETMEGATVPLKMSDFTYVVEDMAGNITYTPVT
KLLEGHSNKPEQDGSDQAPDKKPEAKPEQDGSGQTPDKKKETKPEKDSSG
QTPGKTPQKGQSSRTLEKRSSKRALATK

### GBS322.

GBS322 refers to a surface immunogenic protein, also referred to as ‘sip’. Nucleotide and amino acid sequences of GBS322 sequenced from serotype V isolated strain 2603 V/R are set forth in Ref. 86 as SEQ ID NOs 8539 & 8540. The amino acid sequence is SEQ ID NO: 5 herein:

MNKKVLLTSTMAASLLSVASVQAQETDTTWTARTVSEVKADLVKQDNKSS
YTVKYGDTLSVISEAMSIDMNVLAKINNIADINLIYPETTLTVTYDQKSH
TATSMKIETPATNAAGQTTATVDLKTNQVSVADQKVSLNTISEGMTPEAA
TTIVSPMKTYSSAPALKSKEVLAQEQAVSQAAANEQVSPAPVKSITSEVP
AAKEEVKPTQTSVSQSTTVSPASVAAETPAPVAKVAPVRTVAAPRVASVK
VVTPKVETGASPEHVSAPAVPVTTTSPATDSKLQATEVKSVPVAQKAPTA
TPVAQPASTTNAVAAHPENAGLQPHVAAYKEKVASTYGVNEFSTYRAGDP
GDHGKGLAVDFIVGTNQALGNKVAQYSTQNMAANNISYVIWQQKFYSNTN
SIYGPANTWNAMPDRGGVTANHYDHVHVSFNK

GBS322 contains a N-terminal leader or signal sequence region which is indicated by the underlined sequence near the beginning of SEQ ID NO: 5. One or more amino acids from the leader or signal sequence region of GBS322 may be removed. An example of such a GBS322 fragment is set forth below as SEQ ID NO: 17:

DLVKQDNKSSYTVKYGDTLSVISEAMSIDMNVLAKINNIADINLIYPETT
LTVTYDQKSHTATSMKIETPATNAAGQTTATVDLKTNQVSVADQKVSLNT
ISEGMTPEAATTIVSPMKTYSSAPALKSKEVLAQEQAVSQAAANEQVSPA
PVKSITSEVPAAKEEVKPTQTSVSQSTTVSPASVAAETPAPVAKVAPVRT
VAAPRVASVKVVTPKVETGASPEHVSAPAVPVTTTSPATDSKLQATEVKS
VPVAQKAPTATPVAQPASTTNAVAAHPENAGLQPHVAAYKEKVASTYGVN
EFSTYRAGDPGDHGKGLAVDFIVGTNQALGNKVAQYSTQNMAANNISYVI
WQQKFYSNTNSIYGPANTWNAMPDRGGVTANHYDHVHVSFNK

### Non-GBS Antigens

Where a composition includes one or more GBS antigens, it may also include further antigens from sexually-transmitted pathogens, such as: herpesvirus; N. gonorrhoeae; papillomavirus; C. trachomatis; a hepatitis virus (HAV, HBV, HCV, etc.); a papillomavirus, such as one more of HPV types 6, 11, 16 and 18 (e.g. a L1 virus-like particle from HPV); Treponema pallidum; Haemophilus ducreyi; etc.

Another type of preferred composition includes further antigens that affect the elderly and/or the immunocompromised, and so GBS antigens can be combined with one or more antigens from the following non-GBS pathogens: influenza virus, Enterococcus faecalis, Staphylococcus aureus, Staphylococcus epidermis, Pseudomonas aeruginosa, Legionella pneumophila, Listeria monocytogenes, Neisseria meningitidis, and parainfluenza virus.

### Activity as Immunopotentiating Adjuvants

The saccharides of the invention have the ability to agonise TLR2 (see below). It has been shown in vitro that TLR2 agonists alone are able to activate T cells [112]. Moreover, conjugation of antigens to TLR2 agonists can increase both antibody and T cell responses [113]. As well as being useful as antigens for eliciting immune responses against themselves, therefore, the zwitterionized saccharides of the invention can be used as adjuvants. Thus they can be used to augment the immune response against a second antigen, wherein the second antigen is not the zwitterionized saccharide. As a result, the immune response against the second antigen is greater than when the second antigen is administered alone. The second antigen will typically be co-administered with the zwitterionized saccharide. Co-administration may be via simple admixing or may involve linking the zwitterionized saccharide and the second antigen e.g. in the form of a covalent conjugate.

Thus the invention provides a pharmaceutical composition comprising (i) a saccharide of the invention, and (ii) a second antigen. Further attributes of such compositions are discussed above.

The invention also provides a method of raising an immune response against an antigen in a patient, comprising the step of administering the antigen to the patient in combination with a saccharide of the invention.

The invention also provides the use of a saccharide of the invention in the manufacture of a medicament for administering to a patient in order to adjuvant the immune response against a second antigen that is administered to the patient.

At the time of filing, it is unclear whether the ability of zwitterionized saccharides of the invention to activate T cells arises from APC activation and subsequent T cell co-stimulation, or whether the saccharides can bind to and activate T cells themselves. Both direct and indirect T cell co-stimulatory properties have been ascribed to TLR2 agonists in the past [114,115].

### Conjugation

As mentioned above, the zwitterionized saccharides of the invention can be used without conjugation. In some embodiments, however, conjugation can be used. For instance, it may be desirable to utilise the TLR2-agonising activity of a zwitterionized saccharide in conjunction with a conjugated carrier. As mentioned above, conjugation of antigens to TLR2 agonists can increase both antibody and T cell responses [113].

Where conjugation is used, preferred carrier proteins are bacterial toxins or toxoids, such as diphtheria toxoid or tetanus toxoid. The CRM 197 mutant of diphtheria toxin [116-118] is a particularly preferred carrier for, as is a tetanus toxoid. Other suitable carrier proteins include the N. meningitidis outer membrane protein [119], synthetic peptides [120,121], heat shock proteins [122,123], pertussis proteins [124,125], cytokines [126], lymphokines [126], hormones [126], growth factors [126], artificial proteins comprising multiple human CD4⁺ T cell epitopes from various pathogen-derived antigens [127] such as N19 [128], protein D from H. influenzae [129,130], pneumococcal surface protein PspA [131], pneumolysin [132], iron-uptake proteins [133], toxin A or B from C. difficile [134], a GBS protein (see above) [89], etc.

Attachment to the carrier is preferably via a —NH₂ group e.g. in the side chain of a lysine residue in a carrier protein, or of an arginine residue. Attachment may also be via a —SH group e.g. in the side chain of a cysteine residue.

It is possible to use more than one carrier protein e.g. to reduce the risk of carrier suppression. Thus different carrier proteins can be used for different GBS serotypes e.g. serotype Ia saccharides might be conjugated to CRM197 while serotype Ib saccharides might be conjugated to tetanus toxoid. It is also possible to use more than one carrier protein for a particular saccharide antigen e.g. serotype III saccharides might be in two groups, with some conjugated to CRM197 and others conjugated to tetanus toxoid. In general, however, it is preferred to use the same carrier protein for all saccharides.

A single carrier protein might carry more than one saccharide antigen [135,136]. For example, a single carrier protein might have conjugated to it saccharides from serotypes Ia and Ib. To achieve this goal, different saccharides can be mixed prior to the conjugation reaction. In general, however, it is preferred to have separate conjugates for each serogroup, with the different saccharides being mixed after conjugation. The separate conjugates may be based on the same carrier.

Conjugates with a saccharide:protein ratio (w/w) of between 1:5 (i.e. excess protein) and 5:1 (i.e. excess saccharide) are preferred. Ratios between 1:2 and 5:1 are preferred, as are ratios between 1:1.25 and 1:2.5. Ratios between 1:1 and 4:1 are also preferred. With longer saccharide chains, a weight excess of saccharide is typical.

Compositions may include a small amount of free carrier. When a given carrier protein is present in both free and conjugated form in a composition of the invention, the unconjugated form is preferably no more than 5% of the total amount of the carrier protein in the composition as a whole, and more preferably present at less than 2% by weight.

After conjugation, free and conjugated saccharides can be separated. There are many suitable methods, including hydrophobic chromatography, tangential ultrafiltration, diafiltration etc. [see also refs. 137 & 138, etc.].

Where the composition of the invention includes a depolymerised oligosaccharide, it is preferred that depolymerisation precedes conjugation.

### General

The term “comprising” encompasses “including” as well as “consisting” e.g. a composition “comprising” X may consist exclusively of X or may include something additional e.g. X+ Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where an antigen is described as being “adsorbed” to an adjuvant, it is preferred that at least 50% (by weight) of that antigen is adsorbed e.g. 50%, 60%, 70%, 80%, 90%, 95%, 98% or more. It is preferred that diphtheria toxoid and tetanus toxoid are both totally adsorbed i.e. none is detectable in supernatant. Total adsorption of HBsAg is also preferred.

In some embodiments of the invention, the zwitterionized saccharide is not a de-N-acetylated saccharide from S. agalactiae. In some embodiments it is not a de-N-acetylated saccharide from S. agalactiae to which a bifunctional linker has been attached.

It will be appreciated that ionisable groups may exist in the neutral form shown in formulae herein, or may exist in charged form e.g. depending on pH. Thus a phosphate group may be shown as —P—O—(OH)₂, this formula is merely representative of the neutral phosphate group, and other charged forms are encompassed by the invention.

Similarly, references herein to cationic and anionic groups should be taken to refer to the charge that is present on that group under physiological conditions e.g. where an amine —NH₂ is protonated to give the cationic —NH₃⁺ group, this protonation is one that occurs at physiological pH. In addition where a carboxyl —COOH is deprotonated to give the anionic —COO⁻ group, this protonation is one that can occur at physiological pH.

Moreover, the invention encompasses salts of the charged forms of molecules of the invention.

Sugar rings can exist in open and closed form and, while closed forms are shown in structural formulae herein, open forms are also encompassed by the invention.

Similarly, the invention encompasses isomeric forms of the molecules of the invention, including tautomers (e.g. imine/enamine tautomers), conformers, enantiomers, diastereoisomers, etc.

The invention may involve the introduction of a secondary amine group (—NR¹H₂⁺), a tertiary amine group (—NR¹R²H⁺) or a quaternary amine group (—NR¹R²R³⁺). In a tertiary amine, R¹ and R² can be the same as or different from each other. In a quaternary amine, R¹ R² and R³ can each be the same as or different from each other. The R groups may be electron donating. Potential R groups include C₁₋₈hydrocarbyl, more preferably C₁₋₈alkyl, especially methyl. R is preferably —CH₃, —C₂H₅ or —C₃H₇.

The hydrocarbyl may be substituted with one or more groups, such as: halogen (e.g. Cl, Br, F, I), trihalomethyl, —NO₂, —CN, —N⁺(C₁₋₆alkyl)₂O⁻, —SO₃H, —SOC₁₋₆alkyl, —SO₂C₁₋₆alkyl, —SO₃C₁₋₆alkyl, —OC(═O)OC₁₋₆alkyl, —C(═O)H, —C(═O)C₁₋₆alkyl, —OC(═O)C₁₋₆alkyl, —N(C₁₋₆alkyl)₂, C₁₋₆alkyl, —N(C₁₋₆alkyl)₂, —C(═O)N(C₁₋₆alkyl)₂, —N(C₁₋₆alkyl)C(═O)O(C₁₋₆alkyl), —N(C₁₋₆alkyl)C(═O)N(C₁₋₆ alkyl)₂, —CO₂H, —OC(═O)N(C₁₋₆alkyl)₂, —N(C₁₋₆alkyl)C(═O)C₁₋₆alkyl, —N(C₁₋₆alkyl)C(═S)C₁₋₆alkyl, —N(C₁₋₆alkyl)SO₂N(C₁₋₆alkyl)₂, —CO₂C₁₋₆alkyl, —SO₂N(C₁₋₆alkyl)₂, —C(═O)NH₂, —C(═S)N(C₁₋₆alkyl)₂, —N(C₁₋₆alkyl)SO₂C₁₋₆alkyl, —N(C₁₋₆alkyl)C(═S)N(C₁₋₆alkyl)₂, —NH—C₁₋₆alkyl, —S—C₁₋₆alkyl or —O—C₁₋₆alkyl. The term ‘hydrocarbyl’ includes linear, branched or cyclic monovalent groups consisting of carbon and hydrogen. Hydrocarbyl groups thus include alkyl, alkenyl and alkynyl groups, cycloalkyl (including polycycloalkyl), cycloalkenyl and aryl groups and combinations thereof, e.g. alkylcycloalkyl, alkylpolycycloalkyl, alkylaryl, alkenylaryl, cycloalkylaryl, cycloalkenylaryl, cycloalkylalkyl, polycycloalkylalkyl, arylalkyl, arylalkenyl, arylcycloalkyl and arylcycloalkenyl groups. Preferred hydrocarbyl are C₁₋₁₄ hydrocarbyl, more preferably C₁₋₈ hydrocarbyl.

Where animal (and particularly bovine) materials are used in the culture of cells, they should be obtained from sources that are free from transmissible spongiform encephalopathies (TSEs), and in particular free from bovine spongiform encephalopathy (BSE).

### BRIEF DESCRIPTION OF THE DRAWINGS

FIG. 1 shows the structures of serotypes Ia, Ib, II, III & V of GBS.

FIG. 2 highlights the difference between serotypes Ia and III.

FIG. 3 shows modification of the N-acetyl group on a GlcNAc residue in the GBS-III saccharide.

FIG. 4 shows the structure of N-acetyl neuraminic acid (sialic acid).

FIGS. 5 to 9 show the structures of the repeating units in meningococcal saccharides for: (5) serogroup A; (6) serogroup B; (7) serogroup C; (8) serogroup W135; and (9) serogroup Y.

FIG. 10 highlights the difference between serogroups W135 and Y.

FIG. 11 shows modifications according to the invention.

FIG. 12 shows the repeating unit in the S. pneumoniae type 1 saccharide, taken from ref. 1.

FIG. 13 shows the results of a T cell proliferation assay using PBMCs.

FIG. 14 shows the up-regulation of CD25/CD69 on CD4 T cells, using either B. fragilis PSA or a zwitterionized GBS-III saccharide.

FIG. 15 shows results of a 3H-Thy incorporation assay. For each of the three GBS serotypes (Ia, Ib, III) the four bars are, from left to right: medium; native saccharide; second derivatisation; first derivatisation.

FIG. 16 shows flow cytometry results in relation to MHC class II and CD80 expression. Numbers indicate the percentage of cells present in a window of MHC II-CD80 high expression. From left to right, the seven bars at the two time points are: medium; native Ia; native Ib; native III; modified Ia; modified Ib; modified III.

FIG. 17 shows TNF-α induction by saccharides. Open symbols are native saccharides: ⋄=Ia; □=Ib; Δ=III; filled symbols (♦, ▪, ▴) are the zwitterionized versions. The open circle is medium alone.

FIG. 18 shows the percentage of GFP-positive cells in an assay of TLR2 involvement. For the three saccharide treatments, the five bars show different concentrations used during the incubation, from left to right: 10 μg/ml; 3 μg/ml; 1 μg/ml; 0.3 μg/ml; 0.1 μg/ml.

FIG. 19 compares ³H-Thy incorporation using various saccharides. FIG. 20 shows results with a zwitterionized serotype III saccharide at various doses.

FIG. 21 shows that an anti-TLR2 blocking antibody prevents monocyte activation induced by zwitterionized saccharides, but not by LPS or R848 (resiquimod). The five data groups are, from left to right: medium alone; phenol-extracted zwitterionized Ib; native Ib; resiquimod; LPS. Each group is split into two: left= without anti-TLR2, right= with anti-TLR2.

FIGS. 22 and 23 show that T cell activation by zwitterionized saccharides, at various doses, is dependent on MHC class II expression. Diamonds (⋄ & ♦) show results using B. fragilis PSA, and squares (□ & ▪) show results using zwitterionized GBS-III. Open symbols show results in the presence of anti-MHC-II, whereas closed symbols show results in the absence of anti-MHC-II.

FIG. 24 shows monocyte activation. Open symbols are native saccharides: Δ=Ia; ∘=Ib; □=III; filled symbols (▴, , ▪) are the corresponding zwitterionized versions.

FIG. 25 shows the activity of zwitterionized GBS-Ib saccharide before and after phenol extraction. The five groups are, from left to right: medium alone; GBS-Ib pre-phenol; GBS-Ib post-phenol; native GBS-Ib; and a positive control (synthetic lipopeptide N-palmitoyl-S-[2,3-bis(palmitoyloxy)-(2R,S)-propyl]-(R)-cysteinyl-seryl-(lysyl)(3)-lysine, or “PAMcsk”). The three saccharides were tested at both 6 μg/ml (left in the pair) and 2 μg/ml (right in the pair).

FIG. 26 shows the activity of various GBS-Ib saccharides. From left to right: medium alone; zwitterionized saccharide after phenol extraction; native saccharide; zwitterionized saccharide subjected to reduction to reverse zwitterionization; and the PAMcsk positive control.

FIGS. 27 and 28 show the up-regulation of CD83 (FIG. 27) and CD80/MHC class II (FIG. 28) induced by various saccharides on monocyte-derived dendritic cells. From left to right, the five bars are: medium; phenol-extracted zwitterionized Ib; native Ib; reduced Ib (de-zwitterionized); and endotoxin (LPS).

FIGS. 29 and 30 show the TLR2 dependence of monocyte activation, measured by TNF-a production (FIG. 29) or CD80/MHC II up-regulation (FIG. 30) by purified monocytes. The data are in six groups of columns, from left to right: medium alone; zwitterionized Ib; post-phenol zwitterionized Ib; native Ib; PAMcsk; and anti-TLR2 combinations. The saccharides were tested at 2 μg/ml and 6 μg/ml. The anti-TLR2 group shows data for a combination with the zwitterionized Ib saccharide at both concentrations, and for a combination with PAMcsk.

FIGS. 31 and 32 show the results of assays for DC activation and TNFα production using a new batch of zwitterionized GBS serotype Ib saccharide. In FIG. 31, the groups of data are, from left to right: medium alone; zwitterionized Ib; native Ib; resiquimod; and LPS. In FIG. 32, the groups of data are, from left to right: medium alone; zwitterionized Ib; phenol-extracted zwitterionized Ib; resiquimod; and LPS. In FIG. 32, the left-hand of each pair is without anti-TLR2, and the right-hand of each pair is with anti-TLR2.

FIG. 33 illustrates zwitterionization followed by de-zwitterionization.

FIG. 34 highlights vicinal diols for reduction to aldehydes.

FIG. 35 shows the results of a monocyte activation assay after 24 hours. The y-axis shows % CD80/MHCII (of CD14+ cells). The x-axis shows data for: (1) native GBS Ib saccharide; (2) native GBS III saccharide; (3) zwitterionized GBS Ib saccharide; (4) zwitterionized GBS III saccharide; (5) alternative zwitterionized GBS Ib saccharide; (6) PSA; and (7) PAMcsk. White bars are results using these compounds in the presence of an anti-TLR2 monoclonal antibody; black bars are without the blocking antibody. The “nd” columns indicate that experiments were not done.

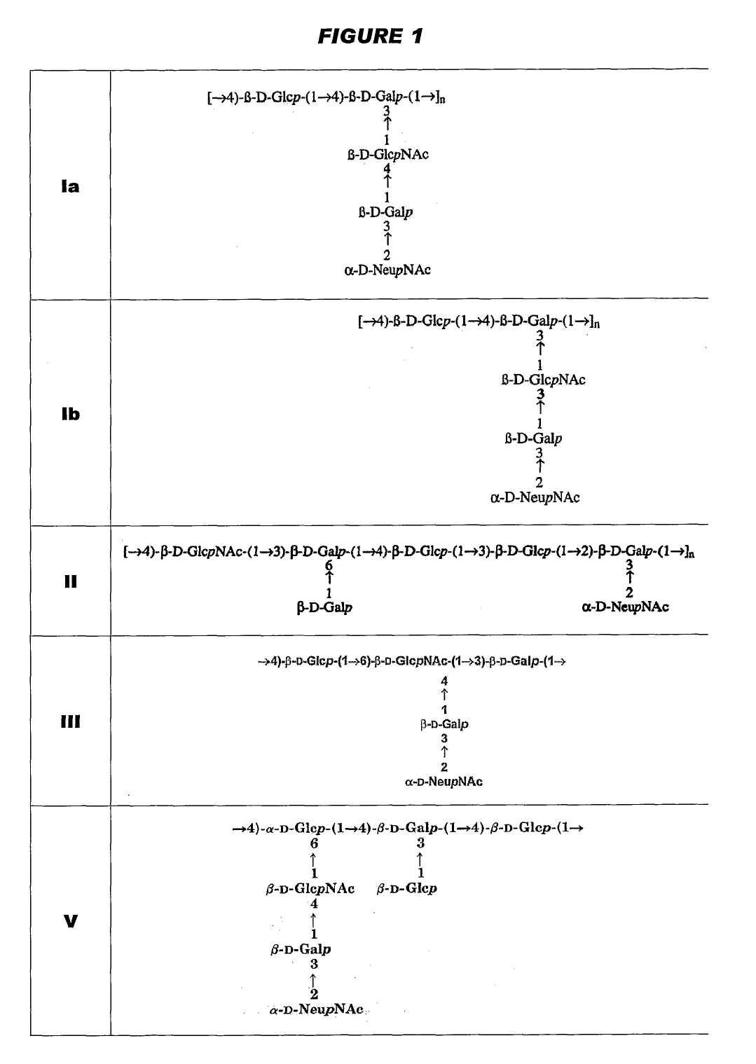

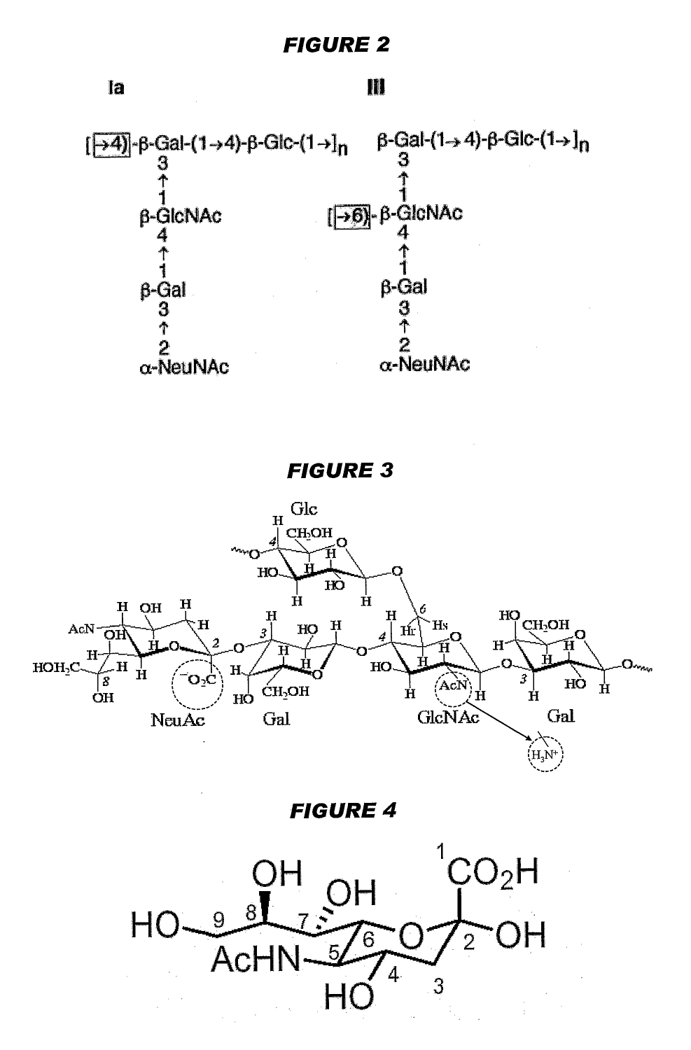

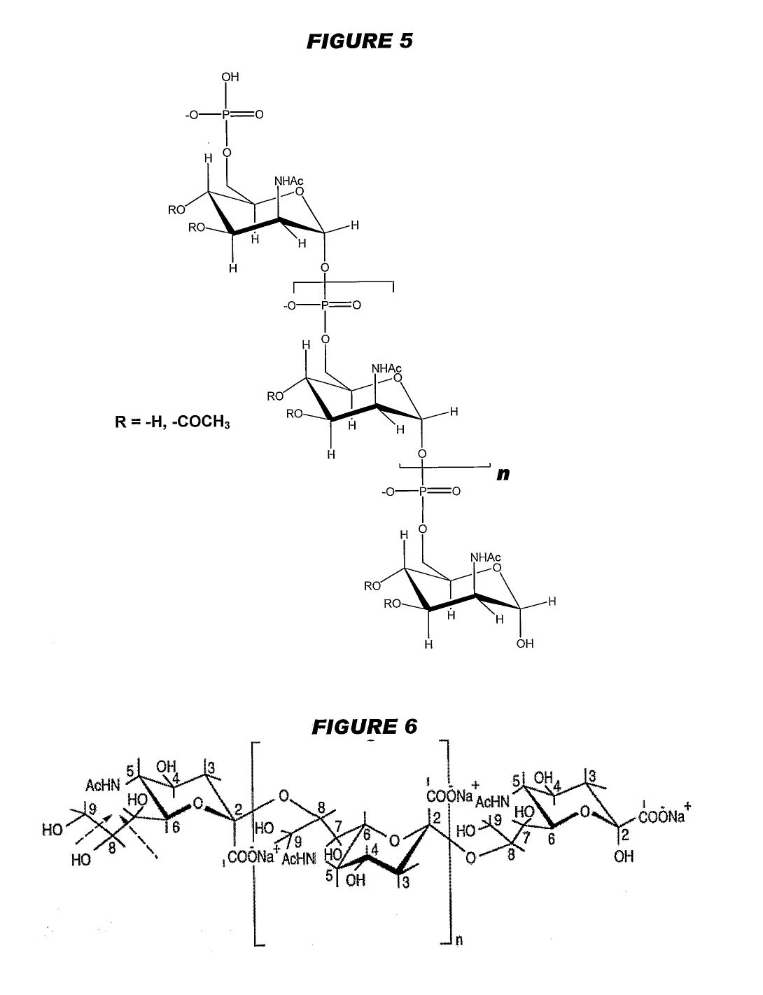

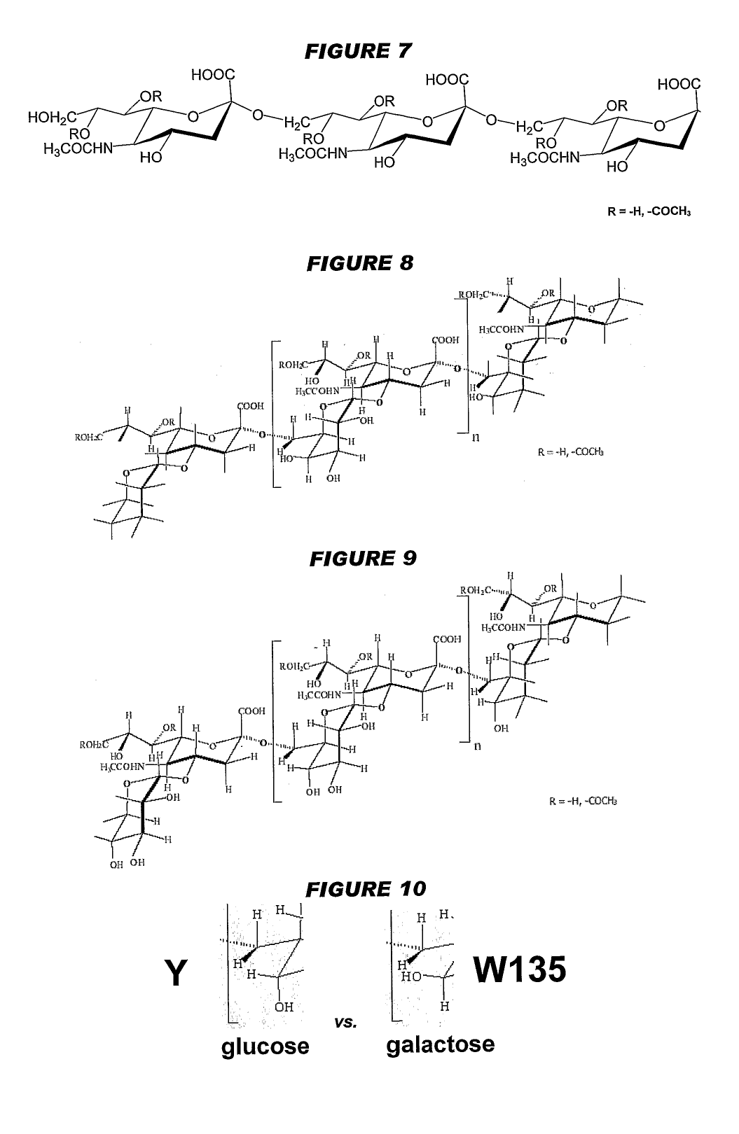

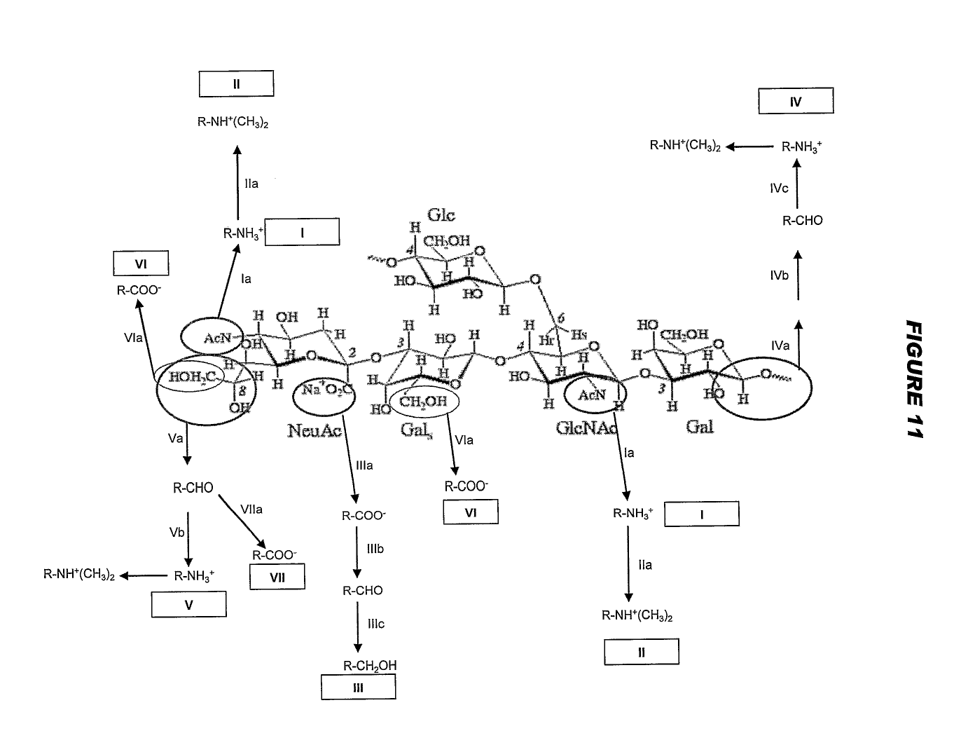

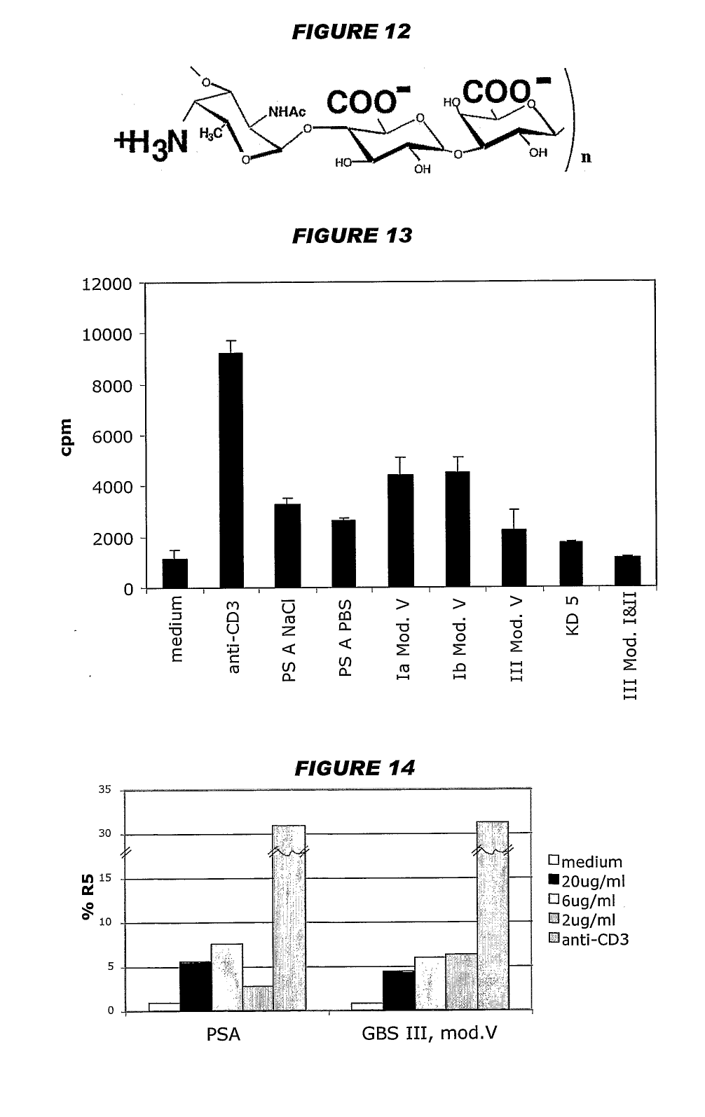

### MODES FOR CARRYING OUT THE INVENTION

### Modification of GBS Saccharides

Capsular saccharides were purified from GBS serotypes Ia, Ib and III by known techniques.

In a first series of derivatisations, the three separate saccharides were treated with sodium periodate to oxidise the terminal —CH₂OH group of the sialic acid residues (shown for serotype III in FIG. 11 as step Va), and were then treated with a mixture of ammonium chloride and sodium cyanoborohydride (step Vb). The resulting amine was then dimethylated (step IIa). Thus the aim was to introduce a cationic group into the sialic acid residues.

In a second series of derivatisations, the saccharides were treated with sodium hydroxide to remove N-acetyl groups from the sialic acid and expose amine groups (step Ia in FIG. 11), and were then treated with formaldehyde to di-methylate the amines (step IIa). Thus the aim was to introduce a cationic group into the sialic acid and/or GlcNAc residues.

In a third series of derivatisations, the saccharides were treated with EDC and sodium borohydride (step III in FIG. 11) to reduce the carboxyl group to an alcohol via an aldehyde. Thus the aim was to remove an anionic group and replace it with a neutral group.

### PBMC Assays

Total human peripheral blood mononuclear cells (PBMCs) were purified from the blood of healthy donors by centrifugation through a Ficoll-Paque cushion. PBMCs were washed and incubated at a concentration of 400,000 cells/well in a 96 well plate in RPMI+Hepes, 5% human AB serum, penicillin, streptomycin and glutamine (medium). The indicated compounds were added at 20 μg/ml. After 6 days of culture, 0.5 μCi of tritiated thymidine (³H-Thy) was added, and proliferation was determined by incorporation of ³H-Thy. Results are shown in FIG. 13 as the mean±standard deviation of duplicate cultures. Positive controls included: (i) medium only, (ii) sterile-filtered PSA (the zwitterionic polysaccharide A from B. fragilis) dissolved in 3M NaCl; (iii) sterile-filtered PSA dissolved in PBS; and (iv) anti-CD3. Negative controls included: (i) the (KD)₅ zwitterionic peptide [2]; and (ii) GBS-III saccharide treated by de-N-acetylation and di-methylation, as described above.

The saccharides from serotypes Ia and Ib, modified by the first derivatisation (i.e. according to scheme V), show good activation of T cells, with higher activity than both the zwitterionic polysaccharide A from B. fragilis and the zwitterioninc (KD)₅ peptide.

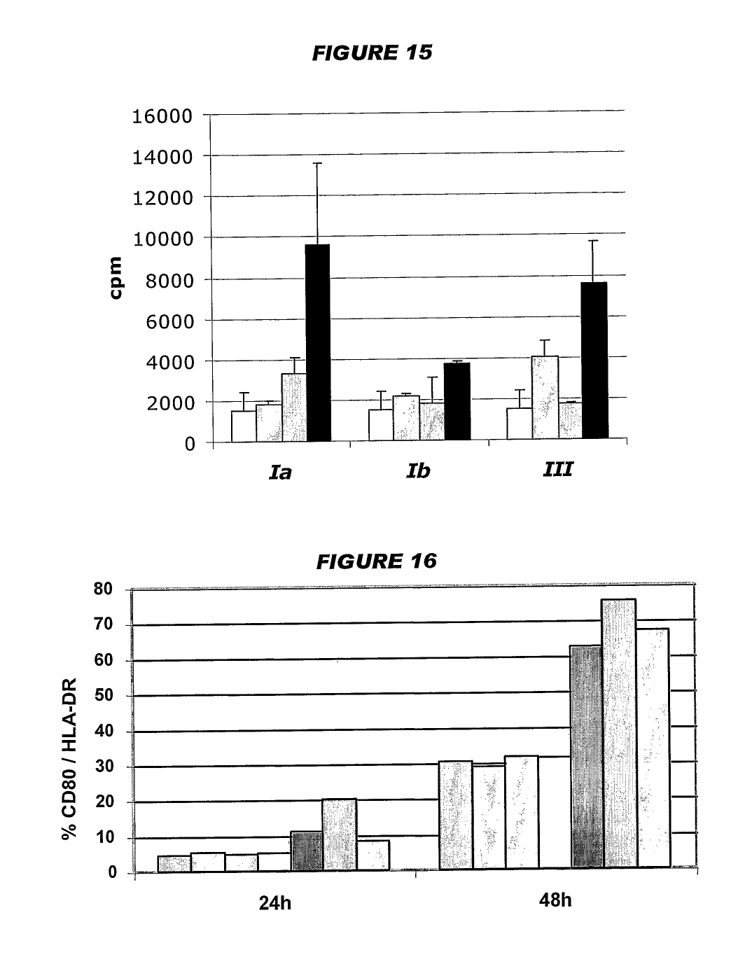

In further experiments, 2.5×10⁵ PBMCs/well were used in a 3H-Thy incorporation assay. Results after 8 days are shown in FIG. 15. For each of GBS serotypes Ia, Ib and III, incorporation of 3H was substantially higher for the saccharide having the first derivatisation (black bars, compared to wild-type in light grey). The second derivatisation gave good results for serotype la, but less so for serotypes Ib and III.

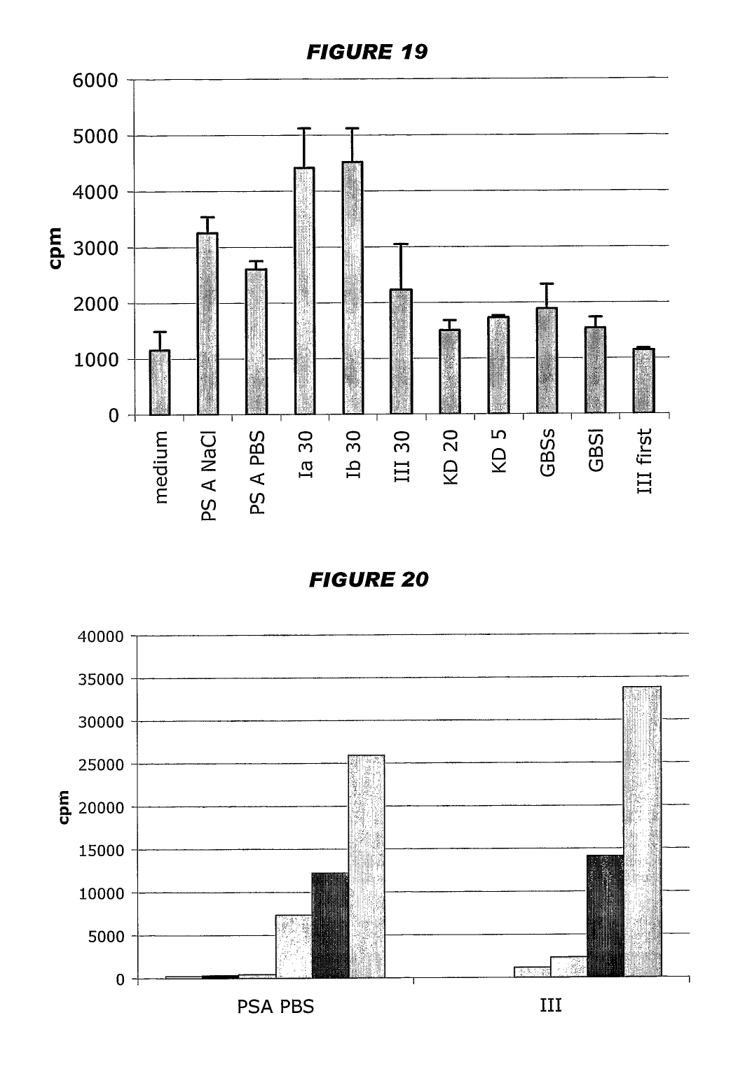

FIG. 19 shows a direct comparison of results from ³H-Thy incorporation assays for: the B. fragilis PSA in saline or PBS; for zwitterionized saccharides from GBS Ia, Ib and III modified according to reaction scheme V (first series of derivatisations); for (KD)₅ and (KD)₂₀ peptides; for unmodified GBS saccharides with short or long chains; and for GBS III modified according to the second series of derivatisations (reaction scheme Ia then IIa).

FIG. 20 shows ³H-Thy incorporation results using the B. fragilis PSA or GBS-III modified according to reaction scheme V. The six bars for each group are, from left to right: 10⁵ monocytes as a negative control; 2×10⁵ T cells as a negative control; and then monocytes+T cells: in culture medium, without saccharide; with the saccharide at 6 μg/ml; with the saccharide at 20 μg/ml; or with anti-CD3 as a positive control.

Further ways of assessing the T cell activating activity of the modified saccharides include, but are not limited to, measurement of activation markers and/or cytokine production after incubation of T cells with the saccharides (and, optionally, antigen presenting cells). Anti-MHC-III antibodies will typically be able to block such activation. Animals treated with the modified saccharides should raise antibodies that recognise the native saccharides, and can be protected against infection.

The effect of the first derivatisation on the ability of the type III saccharide to up-regulate CD25/CD69 on CD4 T cells was investigated. MACS-purified CD14⁺ gamma-irradiated monocytes and negatively MACS-purified T cells were mixed at various ratios, incubated for 6 days with the indicated substances, and analysed by FACS. The results in FIG. 14 show that the zwitterionized saccharide can up-regulate CD25/CD69 in a similar way to PSA from B. fragilis.

Similar experiments were performed in relation to MHC class II and CD80. MACS-purified CD14+ monocytes were incubated for 48 hours with native or modified saccharides for each of GBS serotypes Ia, Ib and III, and MHC class II and CD80 expression were assessed by flow cytometry. As shown in FIG. 16, the modified saccharides induced a much higher up-regulation.

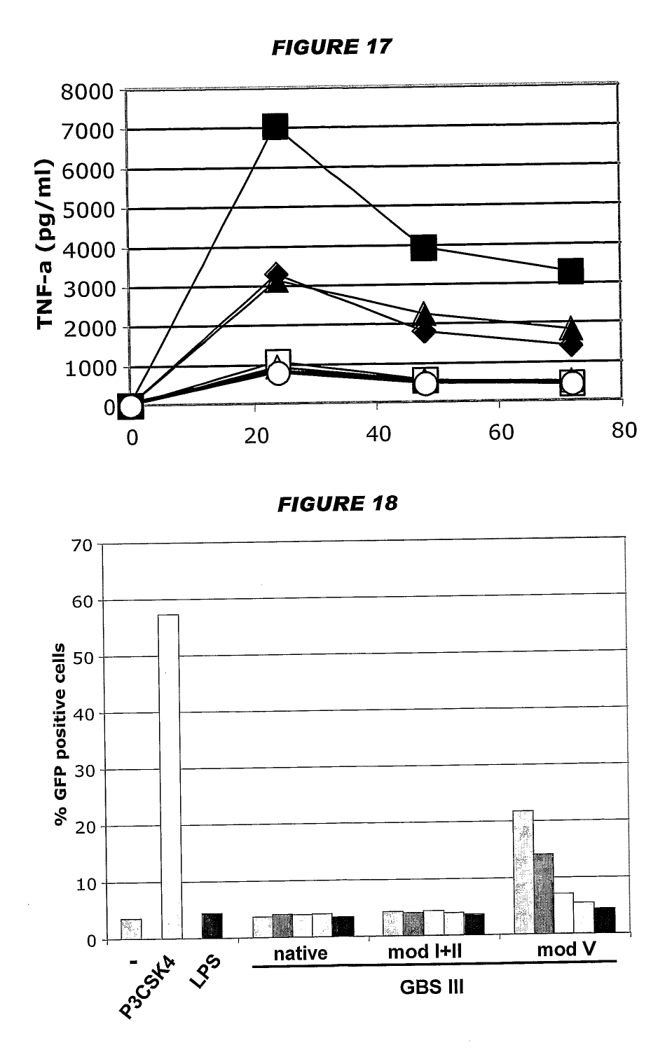

Further experiments with monocytes looked at TNF-α production every 24 hours during a 72 hour incubation with the native and modified saccharides. As shown in FIG. 17, the modified saccharides induced more TNF-a production than the native saccharides.

Further experiments with monocytes measured their activation in terms of up-regulation of MHC class II and CD80, measured by FACS. The results are shown in FIG. 24. Superior activation is seen using the zwitterionized saccharides.

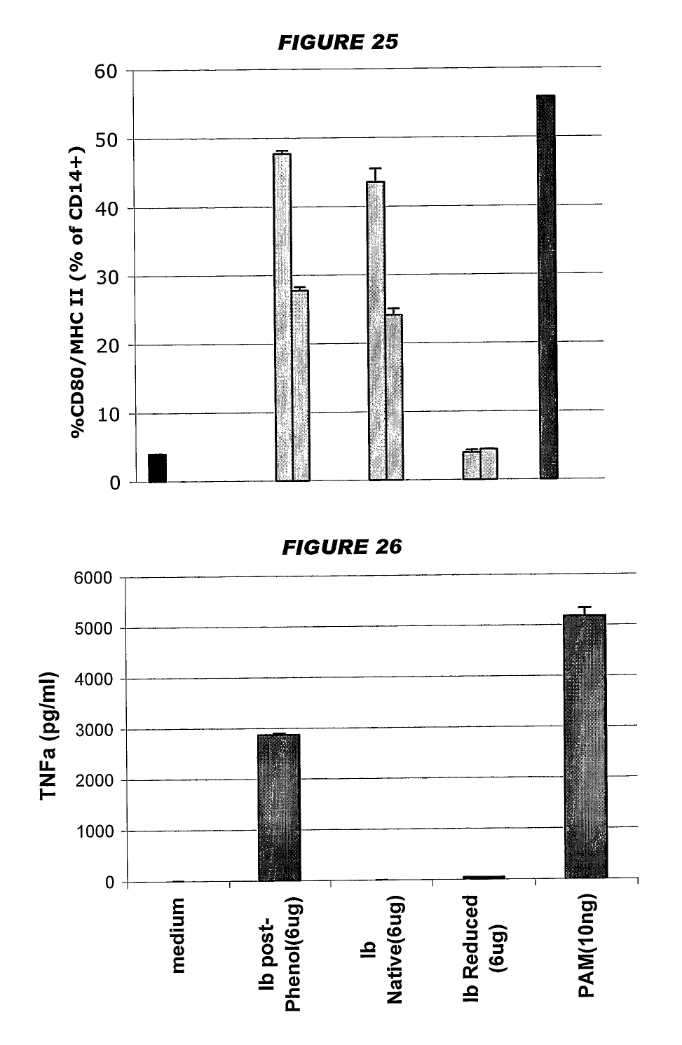

To confirm that monocyte activation was due to the zwitterionized saccharides, rather than being due to another component such as LPS, phenol extraction was performed on the saccharides. As shown in FIG. 25, the phenol extraction did not diminish monocyte activation. In contrast, removal of the introduced negative group after phenol extraction, by use of a reducing agent, removed the activity (FIG. 26).

A separate series of experiments used 293T cells that had been stably transfected with TLR2 and a NFκB-driven GFP reporter construct. These cells were incubated with various ligands: Pam3Cys, a known TLR2 agonist, as a positive control; medium alone, as a negative control; LPS, a TLR4 agonist, as a negative control; and the saccharide from GBS serotype III, either (i) native; (ii) modified by the first derivatisation; or (iii) modified by the second derivatisation. After overnight culture, GFP expression was assessed by flow cytometry. As shown in FIG. 18, the saccharide modified by the first derivatisation showed increased GFP expression.

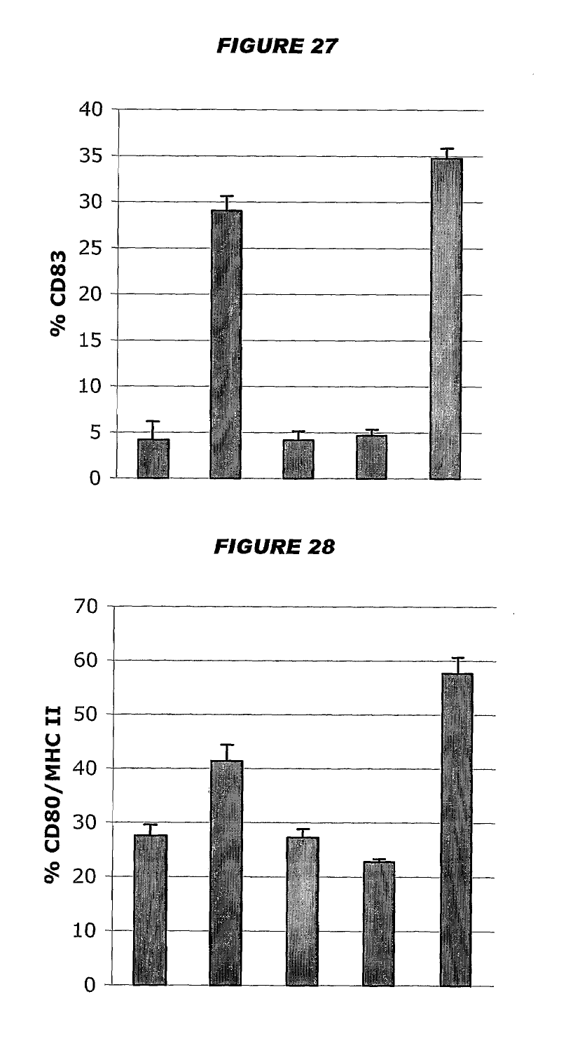

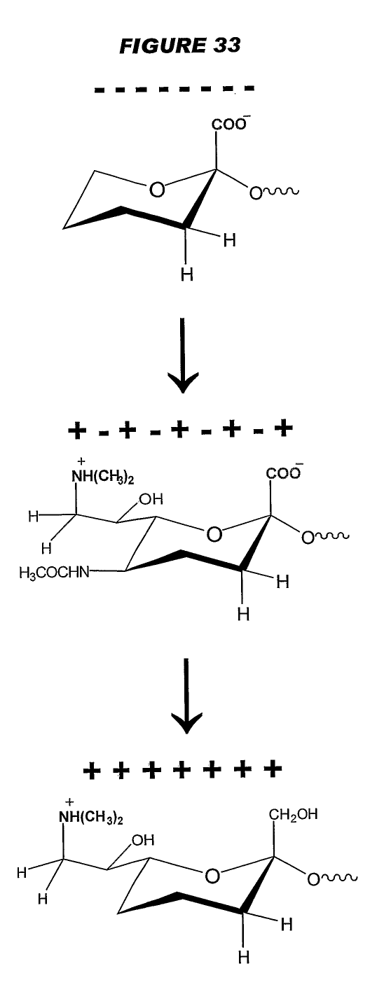

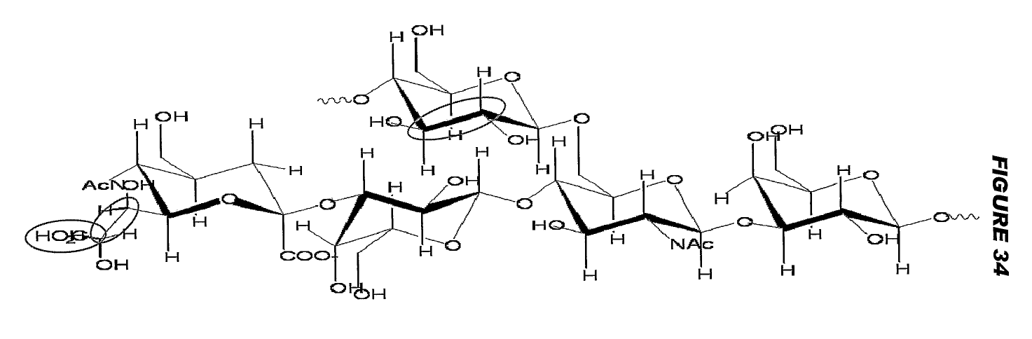

Activation of monocyte-derived dendritic cells (Mo-DCs) was assessed by looking at up-regulation of CD83 (a DC maturation marker), CD80 (a DC activation marker) and MHC class II, as well as by assaying TNF-α production. Results are in FIGS. 27 and 28. The zwitterionized saccharides were able to activate the DCs more effectively than both the native saccharides and the de-zwitterionized saccharides (obtained by treating the zwitterionized saccharides to reduce —COOH to —CHO; FIG. 33). The zwitterionized saccharide was also much more effective than the native saccharide in a TNF-α assay.

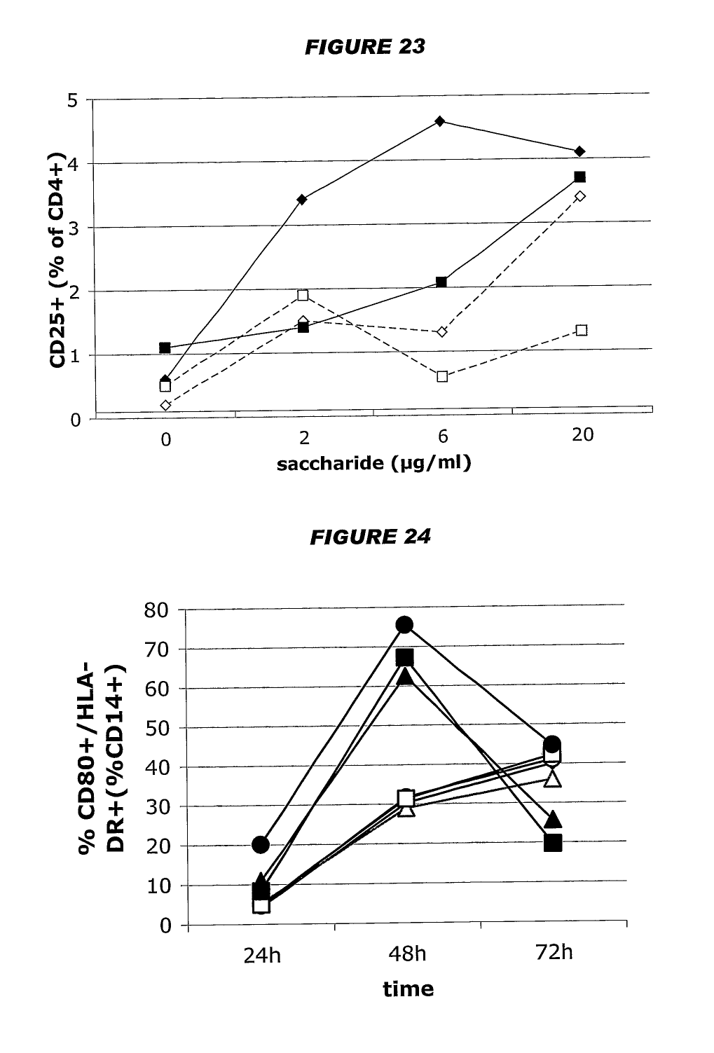

FIGS. 22 and 23 show that the proliferation of T cells induced by the zwitterionized saccharides is dependent on MHC class II. The saccharides were used in a T cell proliferation assay in the absence or presence of anti-MHC-II antibodies. The antibodies inhibited proliferation when present.

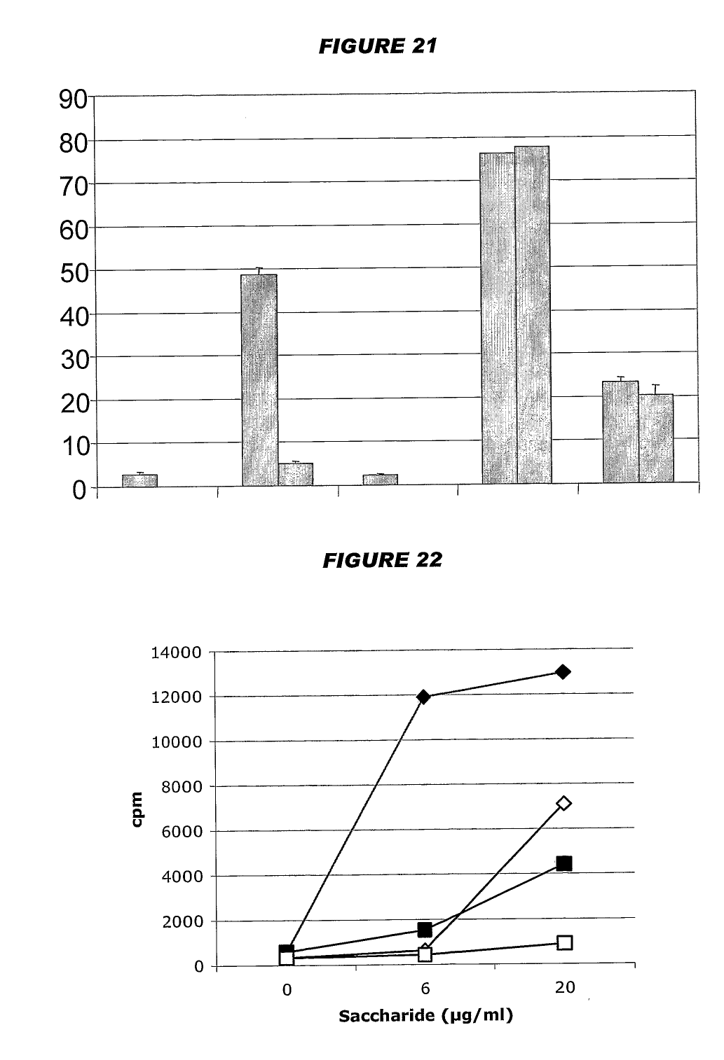

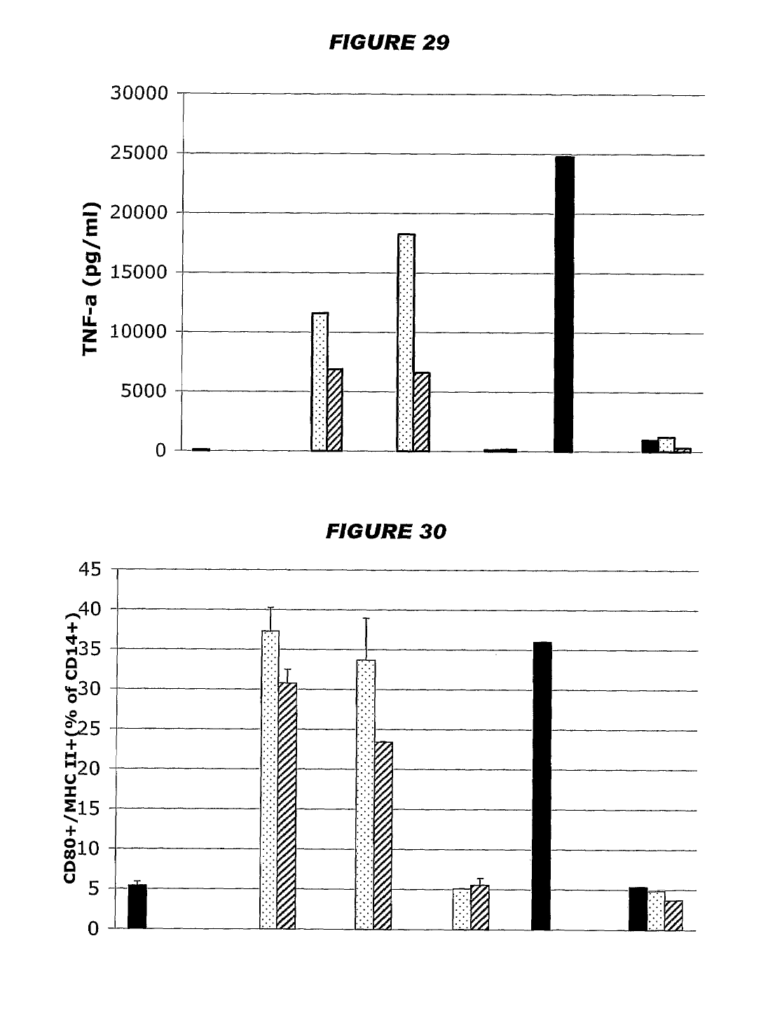

To confirm that monocyte activation by the zwitterionized saccharides is dependent on the presence of a TLR2 receptor, serotype Ib saccharide was used in a TNFα assay (FIG. 29) and a CD80/MHC-II assay (FIG. 30) in the absence or presence of an anti-TLR2 antibody. The antibody was able to block activation by both the zwitterionized saccharides and the PAMcsk positive control. The blocking antibody prevented monocyte activation by the zwitterionized saccharide, but did not block activation by LPS (a TLR4 agonist) or by resiquimod (a TLR7/8 agonist) (FIG. 21).

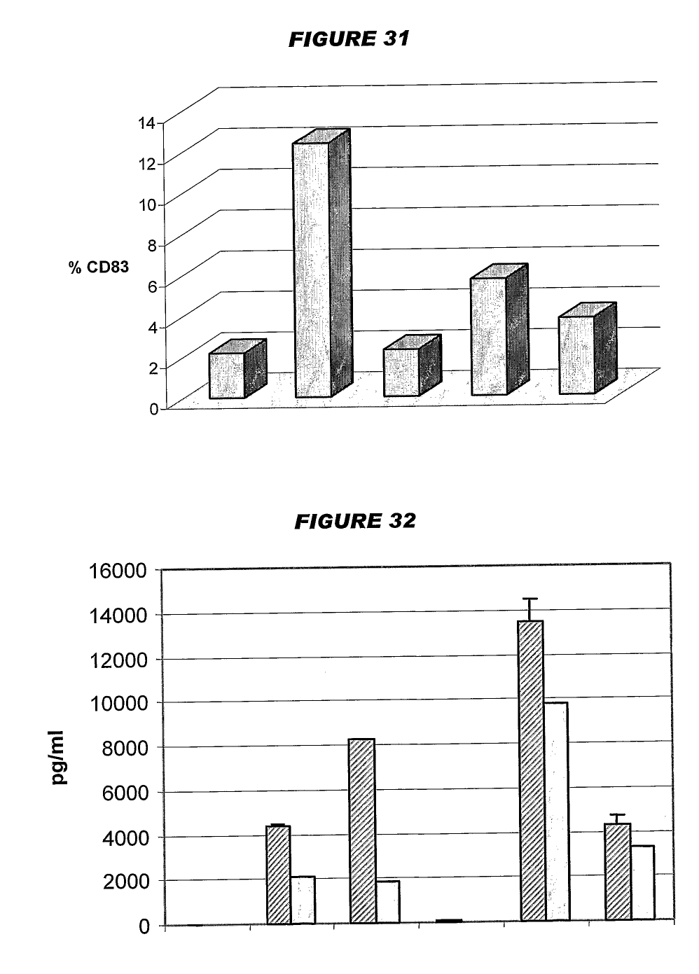

CD83 and TNF-α assays were performed again using a new batch of zwitterionized Ib saccharide, modified according to reaction scheme V in FIG. 11. Results are shown in FIGS. 31 and 32.

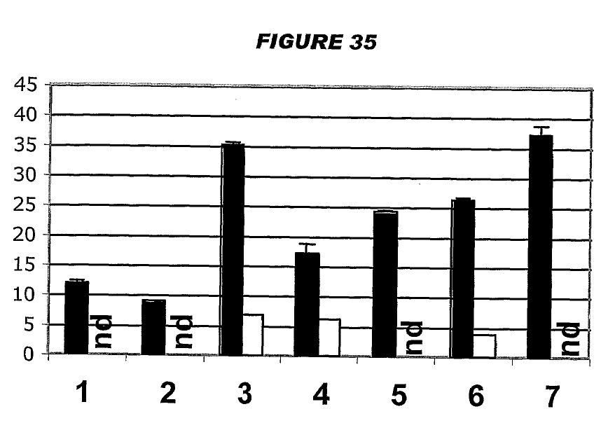

Further confirmation that the zwitterionized saccharides activate APCs in a TLR2-dependent manner comes from FIG. 35. The ability to activate monocytes was blocked by using an anti-TLR2 monoclonal antibody.

### CONCLUSIONS

Naturally-occurring bacterial polysaccharides which contain both positive and negative charges are able to activate T cells and antigen presenting cells (APCs), but the vast majority of bacterial capsular saccharides are anionic and do not have these qualities. Chemical modification to introduce positive charges into anionic saccharides gives them the capacity to activate human monocytes, human monocyte-derived dendritic cells (Mo-DCs) and mouse bone-marrow derived DCs. Cells are induced to up-regulate MHC class II and co-stimulatory molecules and to produce cytokines. In mixed monocyte-T cell co-cultures, the modified saccharides induce MHC II-dependent T-cell proliferation and up-regulation of activation markers. These stimulatory qualities of the modified saccharides are not affected by phenol extraction, and they disappear when the introduced positive charge is chemically removed. The ability of the modified saccharides to activate APCs can be blocked by anti-TLR2 monoclonal antibodies, and TLR2 transfectants show reporter gene transcription upon incubation with the modified saccharides.

It will be understood that the invention has been described by way of example only and modifications may be made whilst remaining within the scope and spirit of the invention.

### REFERENCES

### The Contents of which are Hereby Incorporated by Reference

[1] Kalka-Moll et al. (2002) J Immunol 169:6149-53.
[2] Tzianabos et al. (2000) J Biol Chem 275:6733-40.
[3] WO02/45708.
[4] Wang et al. (2000) PNAS USA 97:13478-83.
[5] Paoletti et al. (1992) J Clin Invest 89:203-9.
[6] Kalka-Moll et al. (2000) J Immunol 164:719-24.
[7] Cobb et al. (2004) Cell 117:677-87.
[8] Paoletti et al. (1990) J Biol Chem 265:18278-83.
[9] Paoletti et al. (1992) J Clin Invest 89:203-9.
[10] Wessels et al. (1987) Proc Natl Acad Sci USA 84:9170-4.
[11] Wang et al. (2003) Vaccine 21:1112-7.
[12] U.S. Pat. Nos. 6,027,733 & 6,274,144.
[13] www.polymer.de
[14] Lewis et al. (2004) PNAS USA 101:11123-8.
[15] Gennaro (2000) Remington: The Science and Practice of Pharmacy. 20th ed., ISBN: 0683306472.
[16] Glezen & Alpers (1999) Clin. Infect. Dis. 28:219-224
[17] Madoff et al. (1994) J Clin Invest 94:286-92.
[18] Paoletti et al. (1994) Infect Immun 62:3236-43.
[19] Vaccine Design . . . (1995) eds. Powell & Newman. ISBN: 030644867X. Plenum.
[20] WO00/23105.
[21] WO90/14837.
[22] U.S. Pat. No. 5,057,540.
[23] WO96/33739.
[24] EP-A-0109942.
[25] WO96/11711.
[26] WO00/07621.
[27] Barr et al. (1998) Advanced Drug Delivery Reviews 32:247-271.
[28] Sjolanderet et al. (1998) Advanced Drug Delivery Reviews 32:321-338.
[29] Niikura et al. (2002) Virology 293:273-280.
[30] Lenz et al. (2001) J Immunol 166:5346-5355.
[31] Pinto et al. (2003) J Infect Dis 188:327-338.
[32] Gerber et al. (2001) Virol 75:4752-4760.
[33] WO03/024480
[34] WO03/024481
[35] Gluck et al. (2002) Vaccine 20:B10-B16.
[36] EP-A-0689454.
[37] Johnson et al. (1999) Bioorg Med Chem Lett 9:2273-2278.
[38] Evans et al. (2003) Expert Rev Vaccines 2:219-229.
[39] Meraldi et al. (2003) Vaccine 21:2485-2491.
[40] Pajak et al. (2003) Vaccine 21:836-842.
[41] Kandimalla et al. (2003) Nucleic Acids Research 31:2393-2400.
[42] WO02/26757.
[43] WO99/62923.
[44] Krieg (2003) Nature Medicine 9:831-835.
[45] McCluskie et al. (2002) FEMS Immunology and Medical Microbiology 32:179-185.
[46] WO98/40100.
[47] U.S. Pat. No. 6,207,646.
[48] U.S. Pat. No. 6,239,116.
[49] U.S. Pat. No. 6,429,199.
[50] Kandimalla et al. (2003) Biochemical Society Transactions 31 (part 3):654-658.
[51] Blackwell et al. (2003) J Immunol 170:4061-4068.
[52] Krieg (2002) Trends Immunol 23:64-65.
[53] WO01/95935.
[54] Kandimalla et al. (2003) BBRC 306:948-953.
[55] Bhagat et al. (2003) BBRC 300:853-861.
[56] WO03/035836.
[57] WO95/17211.
[58] WO98/42375.
[59] Beignon et al. (2002) Infect Immun 70:3012-3019.
[60] Pizzas al. (2001) Vaccine 19:2534-2541.
[61] Pizza et al. (2000) Int J Med Microbiol 290:455-461.
[62] Scharton-Kersten et al. (2000) Infect Immun 68:5306-5313.
[63] Ryan et al. (1999) Infect Immun 67:6270-6280.
[64] Partidos et al. (1999) Immunol Lett 67:209-216.
[65] Peppoloni et al. (2003) Expert Rev Vaccines 2:285-293.
[66] Pine et al. (2002) J Control Release 85:263-270.
[67] Domenighini et al. (1995) Mol Microbiol 15:1165-1167.
[68] WO99/40936.
[69] WO99/44636.
[70] Singh et al. (2001) J Cont Release 70:267-276.
[71] WO99/27960.
[72] U.S. Pat. No. 6,090,406
[73] U.S. Pat. No. 5,916,588
[74] EP-A-0626169.
[75] WO99/52549.
[76] WO01/21207.
[77] WO01/21152.
[78] Andrianov et al. (1998) Biomaterials 19:109-115.
[79] Payne et al. (1998) Adv Drug Delivery Review 31:185-196.
[80] Stanley (2002) Clin Exp Dermatol 27:571-577.
[81] Jones (2003) Curr Opin Investig Drugs 4:214-218.
[82] WO99/11241.
[83] WO94/00153.
[84] WO98/57659.
[85] European patent applications 0835318, 0735898 and 0761231.
[86] WO02/34771.
[87] WO03/093306.
[88] WO2004/018646.
[89] WO2004/041157.
[90] Geysen et al. (1984) PNAS USA 81:3998-4002.
[91] Carter (1994) Methods Mol Biol 36:207-23.
[92] Jameson, B A et al. 1988, CABIOS 4(1):181-186.
[93] Raddrizzani & Hammer (2000) Brief Bioinform 1(2): 179-89.
[94] De Lalla et al. (1999) J. Immunol. 163:1725-29.
[95] Brusic et al. (1998) Bioinformatics 14(2): 121-30
[96] Meister et al. (1995) Vaccine 13(6):581-91.
[97] Roberts et al. (1996) AIDS Res Hum Retroviruses 12(7):593-610.
[98] Maksyutov & Zagrebelnaya (1993) Comput Appl Biosci 9(3):291-7.
[99] Feller & de la Cruz (1991) Nature 349(6311):720-1.
[100] Hopp (1993) Peptide Research 6:183-190.
[101] Welling et al. (1985) FEBS Lett. 188:215-218.
[102] Davenport et al. (1995) Immunogenetics 42:392-297.
[103] Bodanszky (1993) Principles of Peptide Synthesis (ISBN: 0387564314).
[104] Fields et al. (1997) Meth Enzymol 289: Solid-Phase Peptide Synthesis. ISBN: 0121821900.
[105] Chan & White (2000) Fmoc Solid Phase Peptide Synthesis. ISBN: 0199637245.
[106] Kullmann (1987) Enzymatic Peptide Synthesis. ISBN: 0849368413.
[107] Ibba (1996) Biotechnol Genet Eng Rev 13:197-216.
[108] Qi Chen et al. (2002) Infect Immun 70:6409-15.
[109] Beckmann et al. (2002) Infect Immun 70:2869-76.
[110] Cheng et al. (2002) Infect Immun 70:2408-13.
[111] Cheng et al. (2001) Infect Immun 69:2302-8.
[112] Wang et al. (2000) Infect Immun 68:3965-3970.
[113] Jackson et al. (2004) Proc Natl Acad Sci USA 101:15440-15445.
[114] Liu et al. (2006) Proc Natl Acad Sci USA 103:7048-7053.
[115] Cottalorda et al. (2006) Eur J Immunol 36:1684-1693.
[116] Anonymous (January 2002) Research Disclosure, 453077.
[117] Anderson (1983) Infect Immun 39(1):233-238.
[118] Anderson et al. (1985) J Clin Invest 76(1):52-59.
[119] EP-A-0372501.
[120] EP-A-03 78881.
[121] EP-A-0427347.
[122] WO93/17712
[123] WO94/03208.
[124] WO98/58668.
[125] EP-A-0471177.
[126] WO91/01146
[127] Falugi et al. (2001) Eur J Immunol 31:3816-24.
[128] Baraldo et al. (2004) Infect Immun 72:4884-87.
[129] EP-A-0594610.
[130] WO00/56360.
[131] WO02/091998.
[132] Kuo et al. (1995) Infect Immun 63:2706-13.
[133] WO01/72337
[134] WO00/61761.
[135] WO99/42130.
[136] WO2004/011027.
[137] Lei et al. (2000) Dev Biol (Basel) 103:259-264.
[138] WO00/38711; U.S. Pat. No. 6,146,902.

1. A method for modifying a bacterial capsular saccharide antigen, comprising a step of:
(i) if the saccharide is anionic, converting a neutral group in the saccharide into a cationic group;
(ii) if the saccharide is cationic, converting a neutral group in the saccharide into an anionic group;
(iii) if the saccharide is neutral, converting a first neutral group in the saccharide into an anionic group and converting a second neutral group in the saccharide into a cationic group,
thereby providing a modified saccharide.

2. A modified bacterial capsular saccharide, wherein:
(i) the saccharide in its natural form includes repeating units that are cationic, but the saccharide in its modified form includes repeating units that are zwitterionic or anionic;
(ii) the saccharide in its natural form includes repeating units that are anionic, but the saccharide in its modified form includes repeating units that are zwitterionic or cationic;
(iii) the saccharide in its natural form includes repeating units that include either cationic or anionic groups, but the saccharide in its modified form includes repeating units that include both cationic and anionic groups; or
(iv) the saccharide includes a repeating unit that (1) includes both positively-charged and negatively-charged groups but (2) has no overall charge.

3-4. (canceled)

5. The modified saccharide of claim 2, wherein:
(i) the repeating units in the modified saccharide are zwitterionic; or
(ii) the repeating unit has both a free carboxyl group and a free amino group.

6. (canceled)

7. The method of claim 1, wherein the repeating unit has both a free carboxyl group and a free amino group.

8. The method of claim 1, wherein the saccharide is from group B. streptococcus or B. meningococcus.

9. The method of claim 1, wherein a neutral group is converted to a group with a lower pKb value.

10. The method of claim 1, wherein a N-acetyl group is converted to an amino or amine group.

11. The method of claim 1, wherein positive and negative charges are present on different monosaccharide within a repeating unit.

12. The method of claim 11, wherein the positive and negative charges are not on adjacent monosaccharides within the repeating unit.

13. The method of claim 1, wherein at least 50% of the saccharide's repeating units are zwitterionic repeating units.

14. The method of claim 1, wherein the saccharide is a substantially full-length capsular polysaccharide.

15. The method of claim 1, comprising a step of: deacetylating a N-acetyl group on the bacterial capsular saccharide in the presence of a base or enzyme to provide a free amino group.

16. The method of claim 15, further comprising the step of: reacting the free amino group with an aldehyde to provide an amine group.

17. The method of claim 16, wherein the aldehyde is formaldehyde and the amine is a secondary amine.

18. The method of claim 15, wherein the N-acetyl group is present on a NeuAc moiety and/or a GlcNac moiety.

19. The method of claim 1, comprising a step of: reacting a carboxyl group on the bacterial capsular saccharide with pyruvate.

20. The method of claim 19, further comprising the step of: reacting the pyruvate with a carbodiimide or acetic acid.

21. The method of claim 1, comprising the step of: reacting a carboxyl group on the bacterial capsular saccharide with TEMPO (2,2,6,6-tetramethyl-1-piperidine oxoammonium ion) in the presence of hypochlorite and bromide.

22. The method of claim 1, comprising a step of: hydrolysis of a terminal galactose unit on the bacterial capsular saccharide with O3/NO or [beta]-endogalactosidase.

23. The method of claim 22, comprising a step of: oxidizing the terminal galactose unit with galactose oxidase to provide an aldehyde group.

24. The method of claim 23, further comprising the step of: reacting the aldehyde group with a free amino group or an amine group.

25. The method of claim 1, comprising the step of: oxidizing NeuAc groups on the bacterial capsular saccharide to provide aldehyde groups and then reacting the aldehyde groups with a free amino group or an amine group.

26. The method of claim 1, wherein the bacterial capsular saccharide is not from B. fragilis or S. pneumoniae.

27. The modified saccharide of claim 2, wherein the saccharide is from group B. streptococcus or B. meningococcus.

28. The modified saccharide of claim 2, wherein positive and negative charges are present on different monosaccharide within a repeating unit.

29. The modified saccharide of claim 28, wherein the positive and negative charges are not on adjacent monosaccharides within the repeating unit.

30. The modified saccharide of claim 2, wherein at least 50% of the saccharide's repeating units are zwitterionic repeating units.

31. The modified saccharide of claim 2, wherein the saccharide is a substantially full-length capsular polysaccharide.

32. The modified saccharide of claim 2, wherein the bacterial capsular saccharide is not from B. fragilis or S. pneumoniae.


SEQUENCE LISTING (19 sequences)

SEQ ID NO: 1 - protein, 901 aa, Streptococcus agalactiae
PMRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKWTGENGKISYKDLKDGKYQLIEAVSPEDYQKITNKPILTFEVVKGSIKNIIAVNKQISEYHEEGDKHLITNTHIPPKGIIPMTGGKGILSFILIGGAMMSIAGGIYIWKRYKKSSDMSIKKD

SEQ ID NO: 2 - protein, 865 aa, Streptococcus agalactiae
PMRKYQKFSKILTLSLFCLSQIPLNTNVLGESTVPENGAKGKLWKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDWFVLDNSNSMNNDGPNFQRHNKAKKAAEALGTAVKDILGANSDNRVALVTYGSDIFDGRSVDWKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTEIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKWTGENGKISYKDLKDGKYQLIEAVSPEDYQKITNKPILTFEWKGSIKNIIAVNKQISEYHEEGDKHLITNTHIPPKGIIPMTGGKGILS

SEQ ID NO: 3 - protein, 855 aa, Streptococcus agalactiae
PMRKYQKFSKILTLSLFCLSQIPLNTNVLGESTVPENGAKGKLWKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDWKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKWTGENGKISYKDLKDGKYQLIEAVSPEDYQKITNKPILTFEWKGSIKNIIAVNKQISEYHEEGDKHLITNTHIPPKGI

SEQ ID NO: 4 - protein, 553 aa, Streptococcus agalactiae
PMKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLWDALDSKSNVRYLYVEDLKNSPSNITKAYAVPFVLELPVANSTGTGFLSEINIYPKNWTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPATRSGIGTAIFVAIGAAVMAFAVKGMKRRTKD

SEQ ID NO: 5 - protein, 518 aa, Streptococcus agalactiae
PAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTGGIGTAIFVAIGAAVMAFAVKGMKRRTKDN

SEQ ID NO: 6 - protein, 524 aa, Streptococcus agalactiae
PMKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLWDALDSKSNVRYLYVEDLKNSPSNITKAYAVPFVLELPVANSTGTGFLSEINIYPKNWTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTG

SEQ ID NO: 7 - protein, 519 aa, Streptococcus agalactiae
PMKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLWDALDSKSNVRYLYVEDLKNSPSNITKAYAVPFVLELPVANSTGTGFLSEINIYPKNWTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 8 - protein, 483 aa, Streptococcus agalactiae
PAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLWDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPS

SEQ ID NO: 9 - protein, 271 aa, Streptococcus agalactiae
PAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLWDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKG

SEQ ID NO: 10 - protein, 889 aa, Streptococcus agalactiae
PMKKRQKIWRGLSVTLLILSQIPFGILVQGETQDTNQALGKVIVKKTGDNATPLGKATFVLKNDNDKSETSHETVEGSGEATFENIKPGDYTLREETAPIGYKKTDKTWKVKVADNGATIIEGMDADKAEKRKEVLNAQYPKSAIYEDTKENYPLVNVEGSKVGEQYKALNPINGKDGRREIAEGWLSKKITGVNDLDKNKYKIELTVEGKTTVETKELNQPLDWVLLDNSNSMNNERANNSQRALKAGEAVEKLIDKITSNKDNRVALVTYASTIFDGTEATVSKGVADQNGKALNDSVSWDYHKTTFTATTHNYSYLNLTNDANEVNILKSRIPKEAEHINGDRTLYQFGATFTQKALMKANEILETQSSNARKKLIFHVTDGVPTMSYAINFNPYISTSYQNQFNSFLNKIPDRSGILQEDFIINGDDYQIVKGDGESFKLFSDRKVPVTGGTTQAAYRVPQNQLSVMSNEGYAINSGYIYLYWRDYNWVYPFDPKTKKVSATKQIKTHGEPTTLYFNGNIRPKGYDIFTVGIGVNGDPGATPLEAEKFMQSISSKTENYTNVDDTNKIYDELNKYFKTIVEEKHSIVDGNVTDPMGEMIEFQLKNGQSFTHDDYVLVGNDGSQLKNGVALGGPNSDGGILKDVTVTYDKTSQTIKINHLNLGSGQKVVLTYDVRLKDNYISNKFYNTNNRTTLSPKSEKEPNTIRDFPIPKIRDVREFPVLTISNQKKMGEVEFIKVNKDKHSESLLGAKFQLQIEKDFSGYKQFVPEGSDVTTKNDGKIYFKALQDGNYKLYEISSPDGYIEVKTKPWTFTIQNGEVTNLKADPNANKNQIGYLEGNGKHLITNTPKRPPGVFPKTGGIGTIVYILVGSTFMILTICSFRRKQL

SEQ ID NO: 11 - protein, 860 aa, Streptococcus agalactiae
PGETQDTNQALGKVIVKKTGDNATPLGKATFVLKNDNDKSETSHETVEGSGEATFENIKPGDYTLREETAPIGYKKTDKTWKVKVADNGATIIEGMDADKAEKRKEVLNAQYPKSAIYEDTKENYPLVNVEGSKVGEQYKALNPINGKDGRREIAEGWLSKKITGVNDLDKNKYKIELTVEGKTTVETKELNQPLDWVLLDNSNSMNNERANNSQRALKAGEAVEKLIDKITSNKDNRVALVTYASTIFDGTEATVSKGVADQNGKALNDSVSWDYHKTTFTATTHNYSYLNLTNDANEVNILKSRIPKEAEHINGDRTLYQFGATFTQKALMKANEILETQSSNARKKLIFHVTDGVPTMSYAINFNPYISTSYQNQFNSFLNKIPDRSGILQEDFIINGDDYQIVKGDGESFKLFSDRKVPVTGGTTQAAYRVPQNQLSVMSNEGYAINSGYIYLYWRDYNWVYPFDPKTKKVSATKQIKTHGEPTTLYFNGNIRPKGYDIFTVGIGVNGDPGATPLEAEKFMQSISSKTENYTNVDDTNKIYDELNKYFKTIVEEKHSIVDGNVTDPMGEMIEFQLKNGQSFTHDDYVLVGNDGSQLKNGVALGGPNSDGGILKDVTVTYDKTSQTIKINHLNLGSGQKWLTYDVRLKDNYISNKFYNTNNRTTLSPKSEKEPNTIRDFPIPKIRDVREFPVLTISNQKKMGEVEFIKVNKDKHSESLLGAKFQLQIEKDFSGYKQFVPEGSDVTTKNDGKIYFKALQDGNYKLYEISSPDGYIEVKTKPWTFTIQNGEVTNLKADPNANKNQIGYLEGNGKHLITNTPKRPPGVFPKTGGIGTIVYILVGSTFMILTICSFRRKQL

SEQ ID NO: 12 - protein, 850 aa, Streptococcus agalactiae
PMKKRQKIWRGLSVTLLILSQIPFGILVQGETQDTNQALGKVIVKKTGDNATPLGKATFVLKNDNDKSETSHETVEGSGEATFENIKPGDYTLREETAPIGYKKTDKTWKVKVADNGATIIEGMDADKAEKRKEVLNAQYPKSAIYEDTKENYPLVNVEGSKVGEQYKALNPINGKDGRREIAEGWLSKKITGVNDLDKNKYKIELTVEGKTTVETKELNQPLDWVLLDNSNSMNNERANNSQRALKAGEAVEKLIDKITSNKDNRVALVTYASTIFDGTEATVSKGVADQNGKALNDSVSWDYHKTTFTATTHNYSYLNLTNDANEVNILKSRIPKEAEHINGDRTLYQFGATFTQKALMKANEILETQSSNARKKLIFHVTDGVPTMSYAINFNPYISTSYQNQFNSFLNKIPDRSGILQEDFIINGDDYQIVKGDGESFKLFSDRKVPVTGGTTQAAYRVPQNQLSVMSNEGYAINSGYIYLYWRDYNWVYPFDPKTKKVSATKQIKTHGEPTTLYFNGNIRPKGYDIFTVGIGVNGDPGATPLEAEKFMQSISSKTENYTNVDDTNKIYDELNKYFKTIVEEKHSIVDGNVTDPMGEMIEFQLKNGQSFTHDDYVLVGNDGSQLKNGVALGGPNSDGGILKDVTVTYDKTSQTIKINHLNLGSGQKWLTYDVRLKDNYISNKFYNTNNRTTLSPKSEKEPNTIRDFPIPKIRDVREFPVLTISNQKKMGEVEFIKVNKDKHSESLLGAKFQLQIEKDFSGYKQFVPEGSDVTTKNDGKIYFKALQDGNYKLYEISSPDGYIEVKTKPVVTFTIQNGEVTNLKADPNANKNQIGYLEGNGKHLITNT

SEQ ID NO: 13 - protein, 821 aa, Streptococcus agalactiae
PGETQDTNQALGKVIVKKTGDNATPLGKATFVLKNDNDKSETSHETVEGSGEATFENIKPGDYTLREETAPIGYKKTDKTWKVKVADNGATIIEGMDADKAEKRKEVLNAQYPKSAIYEDTKENYPLVNVEGSKVGEQYKALNPINGKDGRREIAEGWLSKKITGVNDLDKNKYKIELTVEGKTTVETKELNQPLDVWLLDNSNSMNNERANNSQRALKAGEAVEKLIDKITSNKDNRVALVTYASTIFDGTEATVSKGVADQNGKALNDSVSWDYHKTTFTATTHNYSYLNLTNDANEVNILKSRIPKEAEHINGDRTLYQFGATFTQKALMKANEILETQSSNARKKLIFHVTDGVPTMSYAINFNPYISTSYQNQFNSFLNKIPDRSGILQEDFIINGDDYQIVKGDGESFKLFSDRKVPVTGGTTQAAYRVPQNQLSVMSNEGYAINSGYIYLYWRDYNWVYPFDPKTKKVSATKQIKTHGEPTTLYFNGNIRPKGYDIFTVGIGVNGDPGATPLEAEKFMQSISSKTENYTNVDDTNKIYDELNKYFKTIVEEKHSIVDGNVTDPMGEMIEFQLKNGQSFTHDDYVLVGNDGSQLKNGVALGGPNSDGGILKDVTVTYDKTSQTIKINHLNLGSGQKWLTYDVRLKDNYISNKFYNTNNRTTLSPKSEKEPNTIRDFPIPKIRDVREFPVLTISNQKKMGEVEFIKVNKDKHSESLLGAKFQLQIEKDFSGYKQFVPEGSDVTTKNDGKIYFKALQDGNYKLYEISSPDGYIEVKTKPWTFTIQNGEVTNLKADPNANKNQIGYLEGNGKHLITNT

SEQ ID NO: 14 - protein, 1133 aa, Streptococcus agalactiae
PMRKKQKLPFDKLAIALISTSILLNAQSDIKANTVTEDTPATEQAVEPPQPIAVSEESRSSKETKTSQTPSDVGETVADDANDLAPQAPAKTADTPATSKATIRDLNDPSHVKTLQEKAGKGAGTVVAVIDAGFDKNHEAWRLTDKTKARYQSKENLEKAKKEHGITYGEWVNDKVAYYHDYSKDGKNAVDQEHGTHVSGILSGNAPSEMKEPYRLEGAMPEAQLLLMRVEIVNGLADYARNYAQAIRDAVNLGAKVINMSFGNAALAYANLPDETKKAFDYAKSKGVSIVTSAGNDSSFGGKPRLPLADHPDYGWGTPAAADSTLTVASYSPDKQLTETATVKTDDHQDKEMPVISTNRFEPNKAYDYAYANRGTKEDDFKDVEGKIALIERGDIDFKDKIANAKKAGAVGVLIYDNQDKGFPIELPNVDQMPAAFISRRDGLLLKDNPPKTITFNATPKVLPTASGTKLSRFSSWGLTADGNIKPDIAAPGQDILSSVANNKYAKLSGTSMSAPLVAGIMGLLQKQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQVTVQTDKVDGKHFALAPKALYETSWQKITIPANSSKQVTVPIDASRFSKDLLAQMKNGYFLEGFVRFKQDPTKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQWKNYNNDLASTLGSTRFEKTRWDGKDKDGKVVANGTYTYRVRYTPISSGAKEQHTDFDVIVDNTTPEVATSATFSTEDSRLTLASKPKTSQPVYRERIAYTYMDEDLPTTEYISPNEDGTFTLPEEAETMEGATVPLKMSDFTYVVEDMAGNITYTPVTKLLEGHSNKPEQDGSDQAPDKKPEAKPEQDGSGQTPDKKKETKPEKDSSGQTPGKTPQKGQSSRTLEKRSSKRALATKASTRDQLPTTNDKDTNRLHLLKLVMTTFFLG

SEQ ID NO: 15 - protein, 1106 aa, Streptococcus agalactiae
PQSDIKANTVTEDTPATEQAVEPPQPIAVSEESRSSKETKTSQTPSDVGETVADDANDLAPQAPAKTADTPATSKATIRDLNDPSHVKTLQEKAGKGAGTVVAVIDAGFDKNHEAWRLTDKTKARYQSKENLEKAKKEHGITYGEWVNDKVAYYHDYSKDGKNAVDQEHGTHVSGILSGNAPSEMKEPYRLEGAMPEAQLLLMRVEIVNGLADYARNYAQAIRDAVNLGAKVINMSFGNAALAYANLPDETKKAFDYAKSKGVSIVTSAGNDSSFGGKPRLPLADHPDYGWGTPAAADSTLTVASYSPDKQLTETATVKTDDHQDKEMPVISTNRFEPNKAYDYAYANRGTKEDDFKDVEGKIALIERGDIDFKDKIANAKKAGAVGVLIYDNQDKGFPIELPNVDQMPAAFISRRDGLLLKDNPPKTITFNATPKVLPTASGTKLSRFSSWGLTADGNIKPDIAAPGQDILSSVANNKYAKLSGTSMSAPLVAGIMGLLQKQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQVTVQTDKVDGKHFALAPKALYETSWQKITIPANSSKQVTVPIDASRFSKDLLAQMKNGYFLEGFVRFKQDPTKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQWKNYNNDLASTLGSTRFEKTRWDGKDKDGKWANGTYTYRVRYTPISSGAKEQHTDFDVIVDNTTPEVATSATFSTEDSRLTLASKPKTSQPVYRERIAYTYMDEDLPTTEYISPNEDGTFTLPEEAETMEGATVPLKMSDFTYWEDMAGNITYTPVTKLLEGHSNKPEQDGSDQAPDKKPEAKPEQDGSGQTPDKKKETKPEKDSSGQTPGKTPQKGQSSRTLEKRSSKRALATKASTRDQLPTTNDKDTNRLHLLKLVMTTFFLG

SEQ ID NO: 16 - protein, 1097 aa, Streptococcus agalactiae
PMRKKQKLPFDKLAIALISTSILLNAQSDIKANTVTEDTPATEQAVEPPQPIAVSEESRSSKETKTSQTPSDVGETVADDANDLAPQAPAKTADTPATSKATIRDLNDPSHVKTLQEKAGKGAGTWAVIDAGFDKNHEAWRLTDKTKARYQSKENLEKAKKEHGITYGEWWDKVAYYHDYSKDGKNAVDQEHGTHVSGILSGNAPSEMKEPYRLEGAMPEAQLLLMRVEIVNGLADYARNYAQAIRDAVNLGAKVINMSFGNAALAYANLPDETKKAFDYAKSKGVSIVTSAGNDSSFGGKPRLPLADHPDYGWGTPAAADSTLTVASYSPDKQLTETATVKTDDHQDKEMPVISTNRFEPNKAYDYAYANRGTKEDDFKDVEGKIALIERGDIDFKDKIANAKKAGAVGVLIYDNQDKGFPIELPNVDQMPAAFISRRDGLLLKDNPPKTITFNATPKVLPTASGTKLSRFSSWGLTADGNIKPDIAAPGQDILSSVANNKYAKLSGTSMSAPLVAGIMGLLQKQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQVTVQTDKVDGKHFALAPKALYETSWQKITIPANSSKQVTVPIDASRFSKDLLAQMKNGYFLEGFVRFKQDPTKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNWWTSEVTEQWKNYNNDLASTLGSTRFEKTRWDGKDKDGKWANGTYTYRVRYTPISSGAKEQHTDFDVIVDNTTPEVATSATFSTEDSRLTLASKPKTSQPVYRERIAYTYMDEDLPTTEYISPNEDGTFTLPEEAETMEGATVPLKMSDFTYWEDMAGNITYTPVTKLLEGHSNKPEQDGSDQAPDKKPEAKPEQDGSGQTPDKKKETKPEKDSSGQTPGKTPQKGQSSRTLEKRSSKRALATK

SEQ ID NO: 17 - protein, 1075 aa, Streptococcus agalactiae
PQSDIKANTVTEDTPATEQAVEPPQPIAVSEESRSSKETKTSQTPSDVGETVADDANDLAPQAPAKTADTPATSKATIRDLNDPSHVKTLQEKAGKGAGTVVAVIDAGFDKNHEAWRLTDKTKARYQSKENLEKAKKEHGITYGEWVNDKVAYYHDYSKDGKNAVDQEHGTHVSGILSGNAPSEMKEPYRLEGAMPEAQLLLMRVEIVNGLADYARNYAQAIRDAVNLGAKVINMSFGNAALAYANLPDETKKAFDYAKSKGVSIVTSAGNDSSFGGKPRLPLADHPDYGWGTPAAADSTLTVASYSPDKQLTETATVKTDDHQDKEMPVISTNRFEPNKAYDYAYANRGTKEDDFKDVEGKIALIERGDIDFKDKIANAKKAGAVGVLIYDNQDKGFPIELPNVDQMPAAFISRRDGLLLKDNPPKTITFNATPKVLPTASGTKLSRFSSWGLTADGNIKPDIAAPGQDILSSVANNKYAKLSGTSMSAPLVAGIMGLLQKQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQVTVQTDKVDGKHFALAPKALYETSWQKITIPANSSKQVTVPIDASRFSKDLLAQMKNGYFLEGFVRFKQDPTKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQWKNYNNDLASTLGSTRFEKTRWDGKDKDGKWANGTYTYRVRYTPISSGAKEQHTDFDVIVDNTTPEVATSATFSTEDSRLTLASKPKTSQPVYRERIAYTYMDEDLPTTEYISPNEDGTFTLPEEAETMEGATVPLKMSDFTYWEDMAGNITYTPVTKLLEGHSNKPEQDGSDQAPDKKPEAKPEQDGSGQTPDKKKETKPEKDSSGQTPGKTPQKGQSSRTLEKRSSKRALATK

SEQ ID NO: 18 - protein, 433 aa, Streptococcus agalactiae
PMNKKVLLTSTMAASLLSVASVQAQETDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVSVADQKVSLNTISEGMTPEAATTIVSPMKTYSSAPALKSKEVLAQEQAVSQAAANEQVSPAPVKSITSEVPAAKEEVKPTQTSVSQSTTVSPASVAAETPAPVAKVAPVRTVAAPRVASVKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGTNQALGNKVAQYSTQNMAANNISYVIWQQKFYSNTNSIYGPANTWNAMPDRGGVTANHYDHVHVSFNK

SEQ ID NO: 19 - protein, 393 aa, Streptococcus agalactiae
PDLVKQDNKSSYTVKYGDTLSVISEAMSIDMNVLAKINNIADINLIYPETTLTVTYDQKSHTATSMKIETPATNAAGQTTATVDLKTNQVSVADQKVSLNTISEGMTPEAATTIVSPMKTYSSAPALKSKEVLAQEQAVSQAAANEQVSPAPVKSITSEVPAAKEEVKPTQTSVSQSTTVSPASIVAAETPAPVAKVAPVRTVAAPRVASVKWTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGTNQALGNKVAQYSTQNMAANNISYVIWQQKFYSNTNSIYGPANTWNAMPDRGGVTANHYDHVHVSFNK